SEQUENCE LISTING (156 sequences)

SEQ ID NO: 1 - DNA, 1178 bp, Artificial Sequence
  artificially synthesized
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 2 - DNA, 507 bp, Artificial Sequence
  artificially synthesized
ctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgac

SEQ ID NO: 3 - DNA, 266 bp, Artificial Sequence
  artificially synthesized
ggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccggga

SEQ ID NO: 4 - DNA, 113 bp, Artificial Sequence
  artificially synthesized
cattttgacacccccataatatttttccagaattaacagtataaattgcatctcttgttcaagagttccctatcactctctttaatcactactcacagtaacctcaactcctg

SEQ ID NO: 5 - DNA, 66 bp, Artificial Sequence
  artificially synthesized
atggacatgcgagttcctgcccaactgcttggccttcttttgctctggctgccaggagccaagtgc

SEQ ID NO: 6 - protein, 22 aa, Artificial Sequence
  artificially synthesized
MDMRVPAQLLGLLLLWLPGAKC

SEQ ID NO: 7 - DNA, 57 bp, Artificial Sequence
  artificially synthesized
atggagtttgggctgagctgggttttcctcgttgctctttttagaggtgtacagtgt

SEQ ID NO: 8 - protein, 19 aa, Artificial Sequence
  artificially synthesized
MEFGLSWVFLVALFRGVQC

SEQ ID NO: 9 - DNA, 207 bp, Artificial Sequence
  artificially synthesized
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 10 - protein, 69 aa, Artificial Sequence
  artificially synthesized
TTTPAPRPPTPAPTIASQPLSLRPEACRPAAGGAVHTRGLDFACDIYIWAPLAGTCGVLLLSLVITLYC

SEQ ID NO: 11 - DNA, 336 bp, Artificial Sequence
  artificially synthesized
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 12 - protein, 112 aa, Artificial Sequence
  artificially synthesized
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 126 bp, Artificial Sequence
  artificially synthesized
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 14 - protein, 42 aa, Artificial Sequence
  artificially synthesized
KRGRKKLLYIFKQPFMRPVQTTQEEDGCSCRFPEEEEGGCEL

SEQ ID NO: 15 - DNA, 399 bp, Artificial Sequence
  artificially synthesized
gccccaaccagctcaagtactaagaagacacagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacc

SEQ ID NO: 16 - protein, 133 aa, Artificial Sequence [sequence omitted]

SEQ ID NO: 17 - DNA, 1530 bp, Artificial Sequence
  artificially synthesized
atctgggagctgaagaaggacgtgtacgtggtggagctggactggtaccccgacgcccccggcgagatggtggtattaacctgcgacaccccagaggaagacggcatcacctggaccctggaccagtcaagcgaggtgctgggtagcggcaagaccctgaccatccaggtgaaggagttcggcgacgccggccagtacacctgccacaagggcggcgaggtgctgagccacagcctactgcttctccacaagaaggaggacggcatctggagcaccgacatcctgaaggaccagaaggagcccaagaacaagaccttcctgcgctgcgaggccaagaattactcaggacgattcacctgctggtggctgaccaccatcagcaccgacctgaccttcagcgtgaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaatgcgagcatcagcgtgcgcgcccaggaccgctactacagcagcagctggagcgagtgggccagcgtgccctgcagcggaggtggcggtggaggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgtctgaacagcagagagactagcttcatcactaacggcagctgcctagccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttccgcatccgcgccgtgaccatcgaccgcgtgatgagctacctgaacgcatca

SEQ ID NO: 18 - protein, 510 aa, Artificial Sequence
  artificially synthesized
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 19 - DNA, 342 bp, Artificial Sequence
  artificially synthesized
aactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 20 - protein, 114 aa, Artificial Sequence
  artificially synthesized
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - DNA, 471 bp, Artificial Sequence
  artificially synthesized
tacttcggcaagctggagagcaagctgagcgtgatccgcaacctgaacgaccaggtgctgttcatcgaccagggcaaccgccccctgttcgaggacatgaccgacagcgactgccgcgacaacgccccccgcaccatcttcatcatcagcatgtacaaggacagccagccccgcggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagcgcagcgtgcccggccacgacaacaagatgcagttcgagagcagcagctacgagggctacttcctggcctgcgagaaggagcgcgacctgttcaagctgatcctgaagaaggaggacgagctgggcgaccgcagcatcatgttcaccgtgcagaacgaggac

SEQ ID NO: 22 - protein, 157 aa, Artificial Sequence
  artificially synthesized
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 23 - DNA, 726 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagc

SEQ ID NO: 24 - protein, 242 aa, Artificial Sequence
  artificially synthesized
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 25 - DNA, 705 bp, Artificial Sequence
  artificially synthesized
caggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 26 - protein, 235 aa, Artificial Sequence
  artificially synthesized
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIK

SEQ ID NO: 27 - DNA, 720 bp, Artificial Sequence
  artificially synthesized
gaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaag

SEQ ID NO: 28 - protein, 240 aa, Artificial Sequence
  artificially synthesized
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 29 - DNA, 57 bp, Artificial Sequence
  artificially synthesized
gccaccaacttcagcctgctgaagcaggccggcgacgtggaggagaaccccggcccc

SEQ ID NO: 30 - protein, 19 aa, Artificial Sequence
  artificially synthesized
ATNFSLLKQAGDVEENPGP

SEQ ID NO: 31 - DNA, 711 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 32 - DNA, 1044 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 33 - DNA, 1380 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - DNA, 3264 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtatctgggagctgaagaaggacgtgtacgtggtggagctggactggtaccccgacgcccccggcgagatggtggtattaacctgcgacaccccagaggaagacggcatcacctggaccctggaccagtcaagcgaggtgctgggtagcggcaagaccctgaccatccaggtgaaggagttcggcgacgccggccagtacacctgccacaagggcggcgaggtgctgagccacagcctactgcttctccacaagaaggaggacggcatctggagcaccgacatcctgaaggaccagaaggagcccaagaacaagaccttcctgcgctgcgaggccaagaattactcaggacgattcacctgctggtggctgaccaccatcagcaccgacctgaccttcagcgtgaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaatgcgagcatcagcgtgcgcgcccaggaccgctactacagcagcagctggagcgagtgggccagcgtgccctgcagcggaggtggcggtggaggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgtctgaacagcagagagactagcttcatcactaacggcagctgcctagccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttccgcatccgcgccgtgaccatcgaccgcgtgatgagctacctgaacgcatca

SEQ ID NO: 35 - DNA, 3600 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtatctgggagctgaagaaggacgtgtacgtggtggagctggactggtaccccgacgcccccggcgagatggtggtattaacctgcgacaccccagaggaagacggcatcacctggaccctggaccagtcaagcgaggtgctgggtagcggcaagaccctgaccatccaggtgaaggagttcggcgacgccggccagtacacctgccacaagggcggcgaggtgctgagccacagcctactgcttctccacaagaaggaggacggcatctggagcaccgacatcctgaaggaccagaaggagcccaagaacaagaccttcctgcgctgcgaggccaagaattactcaggacgattcacctgctggtggctgaccaccatcagcaccgacctgaccttcagcgtgaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaatgcgagcatcagcgtgcgcgcccaggaccgctactacagcagcagctggagcgagtgggccagcgtgccctgcagcggaggtggcggtggaggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgtctgaacagcagagagactagcttcatcactaacggcagctgcctagccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttccgcatccgcgccgtgaccatcgaccgcgtgatgagctacctgaacgcatca

SEQ ID NO: 36 - DNA, 2076 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 37 - DNA, 2412 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagc

SEQ ID NO: 38 - DNA, 2205 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgttacttcggcaagctggagagcaagctgagcgtgatccgcaacctgaacgaccaggtgctgttcatcgaccagggcaaccgccccctgttcgaggacatgaccgacagcgactgccgcgacaacgccccccgcaccatcttcatcatcagcatgtacaaggacagccagccccgcggcatggccgtgaccatcagcgtgaagtgcgagaagatcagcaccctgagctgcgagaacaagatcatcagcttcaaggagatgaacccccccgacaacatcaaggacaccaagagcgacatcatcttcttccagcgcagcgtgcccggccacgacaacaagatgcagttcgagagcagcagctacgagggctacttcctggcctgcgagaaggagcgcgacctgttcaagctgatcctgaagaaggaggacgagctgggcgaccgcagcatcatgttcaccgtgcagaacgaggac

SEQ ID NO: 39 - DNA, 2133 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtgccccaaccagctcaagtactaagaagacacagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacc

SEQ ID NO: 40 - DNA, 3392 bp, Artificial Sequence
  artificially synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactctagaccaccatggagtttgggctgagctgggttttcctcgttgctctttttagaggtgtacagtgtatctgggagctgaagaaggacgtgtacgtggtggagctggactggtaccccgacgcccccggcgagatggtggtattaacctgcgacaccccagaggaagacggcatcacctggaccctggaccagtcaagcgaggtgctgggtagcggcaagaccctgaccatccaggtgaaggagttcggcgacgccggccagtacacctgccacaagggcggcgaggtgctgagccacagcctactgcttctccacaagaaggaggacggcatctggagcaccgacatcctgaaggaccagaaggagcccaagaacaagaccttcctgcgctgcgaggccaagaattactcaggacgattcacctgctggtggctgaccaccatcagcaccgacctgaccttcagcgtgaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaatgcgagcatcagcgtgcgcgcccaggaccgctactacagcagcagctggagcgagtgggccagcgtgccctgcagcggaggtggcggtggaggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgtctgaacagcagagagactagcttcatcactaacggcagctgcctagccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttccgcatccgcgccgtgaccatcgaccgcgtgatgagctacctgaacgcatca

SEQ ID NO: 41 - DNA, 1059 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 42 - DNA, 1395 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 43 - DNA, 2463 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggacatgcgagttcctgcccaactgcttggccttcttttgctctggctgccaggagccaagtgccaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 44 - DNA, 2799 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggacatgcgagttcctgcccaactgcttggccttcttttgctctggctgccaggagccaagtgccaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 45 - DNA, 2478 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggacatgcgagttcctgcccaactgcttggccttcttttgctctggctgccaggagccaagtgcgaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaag

SEQ ID NO: 46 - DNA, 2814 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggacatgcgagttcctgcccaactgcttggccttcttttgctctggctgccaggagccaagtgcgaggtgcagctggtggagagcggaggtggactagtacagcctggtggcagcctacgactgagttgcgccgccagcggcttcaccttcagcgacagctggatccactgggtgcgccaggcccccggcaagggcctggagtgggtggcctggatcagcccctacggcggcagcacctactacgccgacagcgtgaagggccgcttcaccatcagcgccgacaccagcaagaacaccgcctacctgcagatgaacagcctgcgcgccgaggacaccgccgtgtactactgcgcccgccgccactggcccggcggcttcgactactggggccagggcaccctggtgaccgtgagcagcggaggcgggggaagcggcggcggagggtctggaggagggggcagtgacatccagatgacccagagccccagcagcctgagcgccagcgtgggcgaccgcgtgaccatcacctgccgcgccagccaggacgtgagcaccgccgtggcctggtaccagcagaagcccggcaaggcccccaagctgctgatctacagcgccagcttcctgtacagcggcgtgcccagccgcttcagcggcagcggcagcggcaccgacttcaccctgaccatcagcagcctgcagcccgaggacttcgccacctactactgccagcagtacctgtaccaccccgccaccttcggccagggcaccaaggtggagatcaag

SEQ ID NO: 47 - DNA, 2591 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactctagaccaccatggacatgcgagttcctgcccaactgcttggccttcttttgctctggctgccaggagccaagtgccaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 48 - DNA, 1920 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactggctagccatatgggcagcggcgccaccaacttcagcctgctgaagcaggccggcgacgtggaggagaaccccggcccctctagagccaccatggacatgcgagttcctgcccaactgcttggccttcttttgctctggctgccaggagccaagtgccaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 49 - DNA, 2148 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtgccccaaccagctcaagtactaagaagacacagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacc

SEQ ID NO: 50 - DNA, 4263 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacatctagaccaccatggagtttgggctgagctgggttttcctcgttgctctttttagaggtgtacagtgtatctgggagctgaagaaggacgtgtacgtggtggagctggactggtaccccgacgcccccggcgagatggtggtattaacctgcgacaccccagaggaagacggcatcacctggaccctggaccagtcaagcgaggtgctgggtagcggcaagaccctgaccatccaggtgaaggagttcggcgacgccggccagtacacctgccacaagggcggcgaggtgctgagccacagcctactgcttctccacaagaaggaggacggcatctggagcaccgacatcctgaaggaccagaaggagcccaagaacaagaccttcctgcgctgcgaggccaagaattactcaggacgattcacctgctggtggctgaccaccatcagcaccgacctgaccttcagcgtgaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccccccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaatgcgagcatcagcgtgcgcgcccaggaccgctactacagcagcagctggagcgagtgggccagcgtgccctgcagcggaggtggcggtggaggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgtctgaacagcagagagactagcttcatcactaacggcagctgcctagccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttccgcatccgcgccgtgaccatcgaccgcgtgatgagctacctgaacgcatcagttaacggcagcggcgccaccaacttcagcctgctgaagcaggccggcgacgtggaggagaaccccggcccctacgtagccaccatggacatgcgagttcctgcccaactgcttggccttcttttgctctggctgccaggagccaagtgccaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 51 - DNA, 4134 bp, Artificial Sequence
  artificially synthesized
gacgtggtgatgacccagagccccctgagcctgcccgtgacccccggcgagcccgccagcatcagctgccgcagcagccagagcctggtgcacagcaaccgcaacacctacctgcactggtacctgcagaagcccggccagagcccccagctgctgatctacaaggtgagcaaccgcttcagcggcgtgcccgaccgcttcagcggcagcggcagcggcaccgacttcaccctgaagatcagccgcgtggaggccgaggacgtgggcgtgtactactgcagccagaacacccacgtgccccccaccttcggccagggcaccaagctggagatcaagggtggcggtggctcgggaggtggagggtcgggtggcggcggatctcaggtgcagctggtgcagagcggcgccgaggtgaagaagcccggcgccagcgtgaaggtgagctgcaaggccagcggctacaccttcaccgactacgagatgcactgggtgcgccaggcccccggccagggcctggagtggatgggcgccctggaccccaagaccggcgacaccgcctacagccagaagttcaagggccgcgtgaccctgaccgccgacaagagcaccagcaccgcctacatggagctgagcagcctgaccagcgaggacaccgccgtgtactactgcacccgcttctacagctacacctactggggccagggcaccctggtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgtaagctagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatatgggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgtctagaccaccatggagtttgggctgagctgggttttcctcgttgctctttttagaggtgtacagtgtatctgggagctgaagaaggacgtgtacgtggtggagctggactggtaccccgacgcccccggcgagatggtggtattaacctgcgacaccccagaggaagacggcatcacctggaccctggaccagtcaagcgaggtgctgggtagcggcaagaccctgaccatccaggtgaaggagttcggcgacgccggccagtacacctgccacaagggcggcgaggtgctgagccacagcctactgcttctccacaagaaggaggacggcatctggagcaccgacatcctgaaggaccagaaggagcccaagaacaagaccttcctgcgctgcgaggccaagaattactcaggacgattcacctgctggtggctgaccaccatcagcaccgacctgaccttcagcgtgaagagcagccgcggcagcagcgacccccagggcgtgacctgcggcgccgccaccctgagcgccgagcgcgtgcgcggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatccgcgacatcatcaagcccgacccacccaagaacctgcagctgaagcccctgaagaacagccgccaggtggaggtgagctgggagtaccccgacacctggagcaccccccacagctacttcagcctgaccttctgcgtgcaggtgcagggcaagagcaagcgcgagaagaaggaccgcgtgttcaccgacaagaccagcgccaccgtgatctgccgcaagaatgcgagcatcagcgtgcgcgcccaggaccgctactacagcagcagctggagcgagtgggccagcgtgccctgcagcggaggtggcggtggaggcagccgcaacctgcccgtggccacccccgaccccggcatgttcccctgcctgcaccacagccagaacctgctgcgcgccgtgagcaacatgctgcagaaggcccgccagaccctggagttctacccctgcaccagcgaggagatcgaccacgaggacatcaccaaggacaagaccagcaccgtggaggcctgcctgcccctggagctgaccaagaacgagagctgtctgaacagcagagagactagcttcatcactaacggcagctgcctagccagccgcaagaccagcttcatgatggccctgtgcctgagcagcatctacgaggacctgaagatgtaccaggtggagttcaagaccatgaacgccaagctgctgatggaccccaagcgccagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttccgcatccgcgccgtgaccatcgaccgcgtgatgagctacctgaacgcatcagttaacggcagcggcgccaccaacttcagcctgctgaagcaggccggcgacgtggaggagaaccccggcccctctagagccaccatggacatgcgagttcctgcccaactgcttggccttcttttgctctggctgccaggagccaagtgccaggtgcagctggtggagagtggaggtggcgtggtacagcccggccgcagcctgcgcctggactgcaaggccagcggcatcaccttcagcaacagcggcatgcactgggtgcgccaggcccccggcaagggcctggagtgggtggccgtgatctggtacgacggcagcaagcgctactacgccgacagcgtgaagggccgcttcaccatcagccgcgacaacagcaagaacaccctgttcctgcagatgaacagcctgcgcgccgaggacaccgccgtgtactactgcgccaccaacgacgactactggggccagggcaccctggtgaccgtgagcagcggtggcggtggctcgggcggtggtgggtcgggtggcggcggatctgagatcgtgctgacccagagccccgccaccctgagcctgagccccggcgagcgcgccaccctgagctgccgcgccagccagagcgtgagcagctacctggcctggtaccagcagaagccaggacaggctccacgactgctaatctatgacgccagcaaccgcgccaccggcatccccgcccgcttcagcggcagcggcagcggcaccgacttcaccctgaccatcagcagcctggagcccgaggacttcgccgtgtactactgccagcagagcagcaactggccccgcaccttcggccagggcaccaaggtggagatcaag

SEQ ID NO: 52 - DNA, 726 bp, Artificial Sequence
  artificially synthesized
gatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagt

SEQ ID NO: 53 - protein, 242 aa, Artificial Sequence
  artificially synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 54 - DNA, 1395 bp, Artificial Sequence
  artificially synthesized
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - protein, 465 aa, Artificial Sequence
  artificially synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 714 bp, Artificial Sequence
  artificially synthesized
gaggtgcagctggtggagagcggaggcggtctggtgcagcccggaggtagcctacgtctgagctgcgctgcaagcggctactcaatcaccaacgactacgcctggaactgggtgcgccaggcacctggcaagggcctggagtgggtgggctacatcagctacagcggctacaccacctacaaccccagcctgaagagccgcttcaccatcagccgcgacaccagcaagaacaccctgtacctgcagatgaacagcctgcgcgccgaggacaccgccgtgtactactgcgctcgctggactagcggtctggactactggggacagggtactctggtgaccgtgagcagcggtggcggtggctcgggaggtggagggtcgggtggcggcggatctgacatccagatgacccagagccccagcagcctgagcgccagcgtgggcgaccgcgtgaccatcacctgcaaggccagcgacctgatccacaactggctggcctggtaccagcagaagcccggcaaggcccccaagctgctgatctacggcgccaccagcctggagaccggcgtgcccagccgcttcagcggcagcggcagcggcaccgacttcaccctgaccatcagcagcctgcagcccgaggacttcgccacctactactgccagcagtactggaccacccccttcaccttcggccagggcaccaaggtggagatcaag

SEQ ID NO: 57 - protein, 238 aa, Artificial Sequence
  artificially synthesized
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSGGGGSGGGGSGGGGSDIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 58 - DNA, 756 bp, Artificial Sequence
  artificially synthesized
gacattgttatgacgcagagccctgattccctcgccgtgtcactgggcgagagggcgactatcaactgtaaaagcagccaatcactgctctatagccgaaaccagaaaaactacctcgcgtggtaccaacagaaaccagggcagccacctaagctgctcattttttgggcaagtacgcgcgaatctggcgtgcctgacaggttttctgggagtggttttggcaccgattttacattgacaatttccagcttgcaagcagaagatgtcgctgtgtactattgtcaacagtacttcagctatcctcttacgtttgggcaaggaaccaaggtggagatcaaaggtggcggtggctcgggcggtggtgggtcgggtggcggcggatctcaggtccagctcgtccagtccggggctgaggttaagaagccgggtgcttctgtgaaagtgtcatgcaaaacgagccgatatacctttacggaatatacgatacactgggtgaggcaagcgccgggtcagaggttggaatggatcggtgggatcaaccctaataatgggatacctaactataatcagaaatttaaaggccgggttactattaccgtggatacgtccgcgtcaacagcctacatggaactctcctcactcaggagcgaagatactgcggtgtattactgtgcccgccgccgcattgcgtatggatacgatgaagggcacgcgatggactactggggacaaggtacacttgtgacggtctcatca

SEQ ID NO: 59 - protein, 252 aa, Artificial Sequence
  artificially synthesized
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGGSGGGGSGGGGSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 60 - DNA, 111 bp, Artificial Sequence
  artificially synthesized
cggagggaccagaggctgccccccgatgcccacaagccccctgggggaggcagtttccggacccccatccaagaggagcaggccgacgcccactccaccctggccaagatc

SEQ ID NO: 61 - protein, 37 aa, Artificial Sequence
  artificially synthesized
RRDQRLPPDAHKPPGGGSFRTPIQEEQADAHSTLAKI

SEQ ID NO: 62 - protein, 5 aa, Artificial Sequence
  artificially synthesized
NSGMH

SEQ ID NO: 63 - protein, 17 aa, Artificial Sequence
  artificially synthesized
VIWYDGSKRYYADSVKG

SEQ ID NO: 64 - protein, 4 aa, Artificial Sequence
  artificially synthesized
NDDY

SEQ ID NO: 65 - protein, 11 aa, Artificial Sequence
  artificially synthesized
RASQSVSSYLA

SEQ ID NO: 66 - protein, 7 aa, Artificial Sequence
  artificially synthesized
DASNRAT

SEQ ID NO: 67 - protein, 9 aa, Artificial Sequence
  artificially synthesized
QQSSNWPRT

SEQ ID NO: 68 - protein, 5 aa, Artificial Sequence
  artificially synthesized
DSWIH

SEQ ID NO: 69 - protein, 17 aa, Artificial Sequence
  artificially synthesized
WISPYGGSTYYADSVKG

SEQ ID NO: 70 - protein, 9 aa, Artificial Sequence
  artificially synthesized
RHWPGGFDY

SEQ ID NO: 71 - protein, 11 aa, Artificial Sequence
  artificially synthesized
RASQDVSTAVA

SEQ ID NO: 72 - protein, 7 aa, Artificial Sequence
  artificially synthesized
SASFLYS

SEQ ID NO: 73 - protein, 9 aa, Artificial Sequence
  artificially synthesized
QQYLYHPAT

SEQ ID NO: 74 - protein, 5 aa, Artificial Sequence
  artificially synthesized
EYTIH

SEQ ID NO: 75 - protein, 17 aa, Artificial Sequence
  artificially synthesized
NINPNNGGTTYNQKFED

SEQ ID NO: 76 - protein, 6 aa, Artificial Sequence
  artificially synthesized
GWNFDY

SEQ ID NO: 77 - protein, 11 aa, Artificial Sequence
  artificially synthesized
KASQDVGTAVD

SEQ ID NO: 78 - protein, 7 aa, Artificial Sequence
  artificially synthesized
WASTRHT

SEQ ID NO: 79 - protein, 9 aa, Artificial Sequence
  artificially synthesized
QQYNSYPLT

SEQ ID NO: 80 - protein, 5 aa, Artificial Sequence
  artificially synthesized
DYEMH

SEQ ID NO: 81 - protein, 17 aa, Artificial Sequence
  artificially synthesized
ALDPKTGDTAYSQKFKG

SEQ ID NO: 82 - protein, 6 aa, Artificial Sequence
  artificially synthesized
FYSYTY

SEQ ID NO: 83 - protein, 16 aa, Artificial Sequence
  artificially synthesized
RSSQSLVHSNRNTYLH

SEQ ID NO: 84 - protein, 7 aa, Artificial Sequence
  artificially synthesized
KVSNRFS

SEQ ID NO: 85 - protein, 9 aa, Artificial Sequence
  artificially synthesized
SQNTHVPPT

SEQ ID NO: 86 - protein, 5 aa, Artificial Sequence
  artificially synthesized
DYGVS

SEQ ID NO: 87 - protein, 16 aa, Artificial Sequence
  artificially synthesized
VIWGSETTYYNSALKS

SEQ ID NO: 88 - protein, 12 aa, Artificial Sequence
  artificially synthesized
HYYYGGSYAMDY

SEQ ID NO: 89 - protein, 11 aa, Artificial Sequence
  artificially synthesized
RASQDISKYLN

SEQ ID NO: 90 - protein, 7 aa, Artificial Sequence
  artificially synthesized
HTSRLHS

SEQ ID NO: 91 - protein, 9 aa, Artificial Sequence
  artificially synthesized
QQGNTLPYT

SEQ ID NO: 92 - protein, 18 aa, Artificial Sequence
  artificially synthesized
EGRGSLLTCGDVEENPGP

SEQ ID NO: 93 - protein, 6 aa, Artificial Sequence
  artificially synthesized
NDYAWN

SEQ ID NO: 94 - protein, 16 aa, Artificial Sequence
  artificially synthesized
YISYSGYTTYNPSLKS

SEQ ID NO: 95 - protein, 7 aa, Artificial Sequence
  artificially synthesized
WTSGLDY

SEQ ID NO: 96 - protein, 11 aa, Artificial Sequence
  artificially synthesized
KASDLIHNWLA

SEQ ID NO: 97 - protein, 7 aa, Artificial Sequence
  artificially synthesized
GATSLET

SEQ ID NO: 98 - protein, 9 aa, Artificial Sequence
  artificially synthesized
QQYWTTPFT

SEQ ID NO: 99 - protein, 5 aa, Artificial Sequence
  artificially synthesized
EYTIH

SEQ ID NO: 100 - protein, 17 aa, Artificial Sequence
  artificially synthesized
GINPNNGIPNYNQKFKG

SEQ ID NO: 101 - protein, 15 aa, Artificial Sequence
  artificially synthesized
RRIAYGYDEGHAMDY

SEQ ID NO: 102 - protein, 17 aa, Artificial Sequence
  artificially synthesized
KSSQSLLYSRNQKNYLA

SEQ ID NO: 103 - protein, 7 aa, Artificial Sequence
  artificially synthesized
WASTRES

SEQ ID NO: 104 - protein, 9 aa, Artificial Sequence
  artificially synthesized
QQYFSYPLT

SEQ ID NO: 105 - protein, 115 aa, Artificial Sequence
  artificially synthesized
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 106 - protein, 112 aa, Artificial Sequence
  artificially synthesized
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 107 - protein, 115 aa, Artificial Sequence
  artificially synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 108 - protein, 107 aa, Artificial Sequence
  artificially synthesized
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 109 - protein, 113 aa, Artificial Sequence [sequence omitted]

SEQ ID NO: 110 - protein, 107 aa, Artificial Sequence [sequence omitted]

SEQ ID NO: 111 - protein, 118 aa, Artificial Sequence [sequence omitted]

SEQ ID NO: 112 - protein, 107 aa, Artificial Sequence [sequence omitted]

SEQ ID NO: 113 - protein, 120 aa, Artificial Sequence
  artificially synthesized
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 114 - protein, 107 aa, Artificial Sequence
  artificially synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 115 - protein, 116 aa, Artificial Sequence
  artificially synthesized
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 116 - protein, 107 aa, Artificial Sequence
  artificially synthesized
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 117 - protein, 124 aa, Artificial Sequence
  artificially synthesized
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 118 - protein, 113 aa, Artificial Sequence
  artificially synthesized
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 119 - protein, 119 aa, Artificial Sequence [sequence omitted]

SEQ ID NO: 120 - protein, 107 aa, Artificial Sequence [sequence omitted]

SEQ ID NO: 121 - protein, 120 aa, Artificial Sequence [sequence omitted]

SEQ ID NO: 122 - protein, 107 aa, Artificial Sequence [sequence omitted]

SEQ ID NO: 123 - protein, 122 aa, Artificial Sequence
  artificially synthesized
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 124 - protein, 107 aa, Artificial Sequence
  artificially synthesized
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 125 - protein, 528 aa, Artificial Sequence
  artificially synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGEAAAKEAAAKEAAAKAGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 126 - protein, 507 aa, Artificial Sequence
  artificially synthesized
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSGGGEAAAKEAAAKEAAAKAGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 127 - protein, 5 aa, Artificial Sequence
  artificially synthesized
GYTMN

SEQ ID NO: 128 - protein, 17 aa, Artificial Sequence
  artificially synthesized
LINPYKGVSTYNQKFKD

SEQ ID NO: 129 - protein, 13 aa, Artificial Sequence
  artificially synthesized
SGYYGDSDWYFDV

SEQ ID NO: 130 - protein, 11 aa, Artificial Sequence
  artificially synthesized
RASQDIRNYLN

SEQ ID NO: 131 - protein, 7 aa, Artificial Sequence
  artificially synthesized
YTSRLHS

SEQ ID NO: 132 - protein, 9 aa, Artificial Sequence
  artificially synthesized
QQGNTLPWT

SEQ ID NO: 133 - protein, 7 aa, Artificial Sequence
  artificially synthesized
SGDYYWT

SEQ ID NO: 134 - protein, 16 aa, Artificial Sequence
  artificially synthesized
HIYYSGNTNYNPSLKS

SEQ ID NO: 135 - protein, 9 aa, Artificial Sequence
  artificially synthesized
DRVTGAFDI

SEQ ID NO: 136 - protein, 11 aa, Artificial Sequence
  artificially synthesized
QASQDISNYLN

SEQ ID NO: 137 - protein, 7 aa, Artificial Sequence
  artificially synthesized
DASNLET

SEQ ID NO: 138 - protein, 9 aa, Artificial Sequence
  artificially synthesized
QHFDHLPLA

SEQ ID NO: 139 - protein, 5 aa, Artificial Sequence
  artificially synthesized
DTYIH

SEQ ID NO: 140 - protein, 17 aa, Artificial Sequence
  artificially synthesized
RIYPTNGYTRYADSVKG

SEQ ID NO: 141 - protein, 11 aa, Artificial Sequence
  artificially synthesized
WGGDGFYAMDY

SEQ ID NO: 142 - protein, 11 aa, Artificial Sequence
  artificially synthesized
RASQDVNTAVA

SEQ ID NO: 143 - protein, 7 aa, Artificial Sequence
  artificially synthesized
SASFLYS

SEQ ID NO: 144 - protein, 14 aa, Artificial Sequence
  artificially synthesized
QQHYTTPPTFGQGT

SEQ ID NO: 145 - DNA, 129 bp, Artificial Sequence
  artificially synthesized
agtaagaggagcaggctcctgcacagtgactacatgaacatgactccccgccgccccgggcccacccgcaagcattaccagccctatgccccaccacgcgacttcgcagcctatcgctccggcggaggg

SEQ ID NO: 146 - protein, 43 aa, Artificial Sequence
  artificially synthesized
SKRSRLLHSDYMNMTPRRPGPTRKHYQPYAPPRDFAAYRSGGG

SEQ ID NO: 147 - DNA, 162 bp, Artificial Sequence
  artificially synthesized
cacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctc

SEQ ID NO: 148 - protein, 54 aa, Artificial Sequence
  artificially synthesized
HLWRRQWRPRRFSALEQGIHPPQAQSKIEELEQEPEPEPEPEPEPEPEPEPEQL

SEQ ID NO: 149 - DNA, 10 bp, Artificial Sequence
  artificially synthesized
gggaatttcc

SEQ ID NO: 150 - DNA, 11 bp, Artificial Sequence
  artificially synthesized
ggggactttcc

SEQ ID NO: 151 - DNA, 52 bp, Artificial Sequence
  artificially synthesized
gggaatttccggggactttccgggaatttccggggactttccgggaatttcc

SEQ ID NO: 152 - DNA, 68 bp, Artificial Sequence
  artificially synthesized
ggggactttccgcttggggactttccgctggggactttccgctggggactttccgctggggactttcc

SEQ ID NO: 153 - DNA, 32 bp, Artificial Sequence
  artificially synthesized
tagagggtatataatggaagctcgacttccag

SEQ ID NO: 154 - DNA, 46 bp, Artificial Sequence
  artificially synthesized
gggtcgaggtaggcgtgtacggtgggaggcctatataagcagagct

SEQ ID NO: 155 - DNA, 182 bp, Artificial Sequence
  artificially synthesized
agcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

SEQ ID NO: 156 - DNA, 256 bp, Artificial Sequence
  artificially synthesized
ggggactttccgcttggggactttccgctggggactttccgctggggactttccgctggggactttccgaattcagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

(19) United States
(12) Patent Application Publication
(10) Pub. No.: US 2023/0000918 A1
(43) Pub. Date: Jan. 5, 2023

(54) Chimeric antigen receptors, compositions and applications thereof

(71) Applicant: SUZHOU NOVA THERAPEUTICS CO. LTD, Suzhou, Jiangsu (CN)

(72) Inventors: Feng WANG, Suzhou, Jiangsu (CN); Hui GUO, Suzhou, Jiangsu (CN); Yan LIU, Suzhou, Jiangsu (CN); Jay ZHANG, Suzhou, Jiangsu (CN)

(21) Appl. No.: 17/781,012

(22) PCT Filed: Nov. 29, 2020
(86) PCT No.: PCT/CN2020/132559

§ 371 Date: May 30, 2022

(30) Foreign Application Priority Data
Nov. 29, 2019 (CN) 201911201134.9
Nov. 29, 2019 (CN) 201911202857.0

(51) Int. Cl. A61K 35/17; A61P 35/00; C07K 14/705; C07K 16/28; C07K 14/54; C07K 16/30; C07K 14/725; C07K 14/55

(52) U.S. Cl. CPC A61K 35/17; A61P 35/00; C07K 14/70578; C07K 14/70575; C07K 16/2818; C07K 16/2827; C07K 14/70517; C07K 14/5434; C07K 16/3069; C07K 14/7051; C07K 14/5443; C07K 14/54; C07K 14/55; C07K 14/70521; C07K 16/303; C07K 14/70532; C07K 2319/33; A61K 2039/5156

(57) ABSTRACT

Provided is application of chimeric antigen receptor (CAR)-modified T (CART) cells in preparing drugs for cancer treatment, the CART cells contain an artificially-introduced costimulatory signal transduction domain, and the CART cell does not contain an artificially-introduced first signal transduction domain.

### TECHNICAL FIELD

The present disclosure belongs to the field of cell therapies, and particularly relates to application of chimeric antigen receptor (CAR)-modified T (CART) cells in preparing drugs for cancer treatment, the CART cells contain artificially-introduced costimulatory signal transduction domains and do not contain an artificially-introduced activating first signal domain.

### BACKGROUND

Chimeric antigen receptor T cells (CART cells) therapy is currently one of the hottest fields of anti-tumor immunotherapy, and is also a major breakthrough in medical immunology in the past decade. CART cells are generally made from patient-derived T-lymphocytes, which are re-engineered in the laboratory to express a tumor antigen-recognizing chimeric receptor together with costimulatory molecules. CART cells are then expanded in vitro and infused back to the patients for tumor targeting and attacking.

Since the development of the first generation of CAR-T in 1989, CAR-T-based cancer immunotherapy has made significant progress. In August 2017, Novartis' CAR-T cell Kymriah was approved by Food and Drug Administration (FDA) of the United States for treatment of relapsed or refractory acute lymphoblastic leukemia (ALL) in patients aged up to 25 years, becoming the world's first autologous cell therapy and marking a milestone in the development history of the CAR-T cell therapy. Two months later, Yescarta, the second CAR-T therapy developed by Kite Pharma Company (acquired by Gilead), was also approved for marketing. It was the first CART therapy approved for the treatment of specific types of non-Hodgkin lymphoma (NHL).

Although the first-generation CD3ζ chain-based CART cells exhibit anti-tumor cytotoxicity by T cell activation, the lack of costimulatory signal, the poor in vivopersistence, and limited secretion of cytokines dampened the long-term anti-tumorefficacy. The second-generation CAR incorporates intracellular domain of a costimulatory signal molecules (CD28 or 4-1BB) on the basis of the first-generation CAR, providing two signals for T cell activation. The co-stimulation of B7/CD28 or 4-1BBL/CD137 in the intracellular signal causes continuous proliferation of T lymphocytes, elevated secretion of IL-2 and IFN-γ, resulting in increased in vivo persistence as well as enhanced anti-tumor efficacy [Dotti G. et al. CD28 costimulation improves expansion and persistence of chimeric antigen receptor modified T cells in lymphoma patients. J Clin Invest, 2011, 121(5): 1822-1826.]. Based on the second-generation CAR, the third-generation CAR further incorporates an additional intracellular domain of costimulatory signal molecule, for example a secondary signal molecule 4-1 BB fused between costimulatory domain CD28 and an ITAM signal chain. The third-generation CAR-engineered T cells exhibit better effector function and in vivo persistence. Different from the previous three generations, the fourth generation is additionally engineered to secrete a transgenic cytokine like IL-12 on the basis of the second generation, also known as T cells redirected for antigen-unrestricted cytokine-initiated killing (TRUCKs for short). TRUCKs not only enhance T cell activation, but also attract and activate innate immune cells, thereby destroying antigen-negative cancer cells. TRUCKs may also be used for therapy of viral infections, metabolic diseases, autoimmune diseases and the like.

The CART cells that are disclosed so far all contain an artificially introduced CD3-mediated first signal. From the second-generation CAR to the fourth-generation CAR, two signals required for T cell activation are combined, thereby bypassing the obstacle that T cells are unable to activate due to the lack of second signal such as B7 on tumor cells, greatly enhancing T cell activation, proliferation, killing ability, and therapeutic efficacy. However, increased costimulatory signals in CAR may reduce the threshold required for effector cell activation, leading to the activation of genetically modified T lymphocyteseven in conditions of a low level of antigens or even without antigen triggering, which could result in the release of high-level cytokines (cytokine storm). In order to solve the risk, researchers introduce suicide systems such as HSV-TK or CD20-Rituximab into the CAR-modified T cells as a break to avoid excessive proliferation of T cells. However, the “waterfall” effect triggered by the off-target activation of CART cells is so fast that these suicide systems may not be able to work in time. Although the scheme of reducing the infusion dosage of CART cells reduced the risk to a certain extent, it also leads to a decline in therapeutic efficacy. Therefore, there is an urgent need in the field to optimize the components of the CAR system to improve the clinical safety of CAR-mediated adoptive cell therapy for tumors.

### SUMMARY

It is generally acknowledged that T cell activation requires the stimulation of two signals, namely two signals related to T cell activation. Among them, the TCR-CD3 complex on the surface of T cells binds to the antigen peptide-major histocompatibility complex (MHC) molecule to provide the first signal of T cell activation, while the costimulatory molecule (such as CD28) on the surface of T cells binds to its corresponding ligand (such as B7), providing the second signal for T cell activation, which is essential for T cell proliferation and survival. Other costimulatory molecules such as 4-1 BB, OX40, GITR, CD40, ICOS, CD152 (CTLA4), CD223(LAG3), CD273(PD-L2), CD274(PD-L1), NKG2C, NKG2D, SLAMF7, and B7-H3 are also considered to have similar functions to that of CD28. The prevailing view that the loss or decreased expression of the first signal stimulation source (such as MHC) molecules on tumor cells may not effectively provide signals required for T cell activation, resulting in the inability of T cells to effectively activate and proliferate.

Tumor marker recognizing TCR-CD3 complexes naturally occurring on the surface of tumor infiltrating T cells would provide natural primary signal (signal 1) for T cell activation, while these T cells cannot always exert killing activity efficiently due to the immunosuppressive microenvironment within tumor tissues. These tumor-recognizing T cells are also found in peripheral blood. Current CARs of CART cells all contain artificially introduced first signal molecules such as scFv-CD3zeta, and the artificially introduced costimulatory molecule signal domains such as scFv-4-1 BB-CD3zeta or scFv-CD28. These tumor markers recognizing CART cells are activated by the first and the second signal, thereby exerting efficient killing activity, meanwhile, the proliferation and survival of the CART cells will further exert the killing activity. However, this mechanism may also cause CART to kill other normal cells expressing the same marker.

It is found by the inventors that: when T cells from peripheral blood of healthy donors are artificially incorporated with CAR containing a costimulatory signal conduction domain (4-1 BB) while without a first signal conduction domain (CD3zeta), these signal 1 lacking CART cells could secret T cell activating factors such as IL-2 and IFN-γ upon co-incubation with tumor cells in vitro, which further support the proliferation and survival of CART cells, indicating that the costimulatory signal of CART cells are activated. In a further experiment with CART cells harboring inducible cytokines expression element, not only secrete cell activating factors like IL-2 and IFN-γ, but also the inducible cytokines were detected in the supernatant. It is inferred that tumor recognition molecules on CAR may activate the costimulatory signal after binding to the tumor antigen, namely the activation of the CART costimulatory signal may be independent of the existence of the first signal.

T cells in peripheral blood from cancer patients contain a small population of T cells that naturally recognize tumor antigens, some of which are derived from tumor infiltrating lymphocytes (TIL). It is further found by the inventors that, when the peripheral blood T cells are artificially engineered with CAR containing the costimulatory signal conduction domain while without the artificially introduced first signal conduction domain, the natural TCR-CD3 complex on the surface of the TIL-derived CART cells specifically recognizes tumor cells (specificity and safety), meanwhile, tumor recognition molecules on CAR could also bind to tumor cells, leading to activation of downstream engineered costimulatory signals, which produce efficient tumor killing and maintain the proliferation and survival of CART cells (effectiveness) in cooperation with the first signal. This design effectively solves the problem of hyperactivation with traditional CART cells (all containing the first signal of T cell activation, namely all or part of the signal domain of CD3, such as CD3zeta, and tumor recognition molecules carried by CAR, such as scFv) to normal tissues which express low levels of tumor related antigen due to the persistence of the first signal and the second signal. In addition to that, although most circulating CART cells that do not recognize the tumor antigens do not possess the first signal required for T cell activation, the tumor recognition molecules carried on its CAR bind to the tumor cells and thus activate the artificially incorporated costimulatory signal. When activated, these CART cells secrete T cell activating factors such as IL-2 and IFN-r which maintain the proliferation and survival of CART cells and further induce the expression of downstream secretory polypeptides (such as aPD-L1 scfv), which could further exert immunomodulatory functions (such as breaking immune tolerance).

Therefore, CART cells of the present disclosure have very important clinical application values: 1) As a delivery carrier that specifically targeting the tumor antigens, CART cells expressed secretory polypeptides to exert immune modulation in the tumor microenvironment; and 2) They effectively solves the overactive immune response of traditional CART cells (all containing the first signal of T cell activation, namely all or part of the signal domain of CD3, such as CD3zeta, and tumor recognition molecules carried by CAR, such as scFv) to normal tissues which express low level of tumor related antigens due to the persistence of the first signal and the second signal.

In one aspect, the present disclosure provides the application of CAR-modified T cells in preparing drugs for cancer treatment. The CART cells include a first CAR, the first CAR includes an antigen binding domain, a transmembrane domain, and an intracellular domain, wherein the intracellular domain is an artificially incorporated costimulatory signal conduction domain, wherein the CART cell does not contain the artificially incorporated first signal conduction domain.

In some embodiments, the T cells are selected from one or more subsets of specific T cells, such as tumor infiltrating lymphocytes (TIL), cytotoxic T lymphocytes (CTL), natural killer T (NKT) cells, δ T cells, or regulatory T cells.

In some embodiments, the CAR includes an antigen binding domain, a transmembrane domain, and an intracellular domain, herein the intracellular domain is the artificially incorporated costimulatory signal transduction domain. In some embodiments, the artificially incorporated costimulatory signal transduction domain is selected from the intracellular signaling domains of one or more costimulatory proteins: 4-1BB (CD137), CD28, CD27, CD30, OX40 , GITR, CD40, ICOS, BAFFR, HVEM, ICAM-1, LCK, CD278(ICOS), CD150(SLAMF1), CD152(CTLA4), CD223(LAG3), CD270(HVEM), CD273(PD-L2), CD274(PD-L1), LAT, NKD2CSLP76, TRIM, ZAP70, DAP-10, DAP-12, LFA-1, CD2, CDS, CD7, CD287, LIGHT, NKG2C, NKG2D, SLAMF7, NKp80, NKp30, NKp44, NKp46, CD160, B7-H3 or ligands that specifically bind to CD83 and the like.

In some embodiments, the artificially introduced costimulatory signal conduction domain is selected from the intracellular signal transduction domain of costimulatory protein 4-1BB. In some embodiments, the artificially introduced costimulatory signal conduction domain is selected from the intracellular signal conduction domain of costimulatory protein OX40. In some embodiments, the artificially introduced costimulatory signal conduction domain is selected from intracellular signal transduction domains of 4-1BB and OX40. In some embodiments, the artificially introduced costimulatory signal conduction domain is selected from intracellular signal conduction domains of 4-1BB and CD28. In some embodiments, the artificially introduced costimulatory signal conduction domain is selected from intracellular signal conduction domains of 4-1BB and LAGS. In some embodiments, the artificially introduced costimulatory signal conduction domain is selected from intracellular signal conduction domains of 4-1BB, CD28 and OX40.

In some embodiments, the intracellular signal transduction domain of 4-1BB has a nucleic acid sequence as shown in SEQ ID NO: 13 or an amino acid sequence as shown in SEQ ID NO: 14. In some embodiments, the intracellular signal transduction domain of OX40 has a nucleic acid sequence as shown in SEQ ID NO: 60 or an amino acid sequence as shown in SEQ ID NO: 61. In some embodiments, the intracellular signal transduction domain of CD28 has a nucleic acid sequence as shown in SEQ ID NO: 145 or an amino acid sequence as shown in SEQ ID NO: 146. In some embodiments, the intracellular signal transduction domain of LAGS has a nucleic acid sequence as shown in SEQ ID NO: 147 or an amino acid sequence as shown in SEQ ID NO: 148.

In some embodiments, the transmembrane domain of the first CAR is derived from the transmembrane region of CD8. In some embodiments, the transmembrane region of CD8 has a nucleic acid sequence as shown in SEQ ID NO: 9 or an amino acid sequence as shown in SEQ ID NO: 10.

In some embodiments, the antigen binding domain of the first CAR binds to disease-associated cell surface antigens.

In some embodiments, the disease-associated cell surface antigen is selected from immune checkpoint proteins.

In some embodiments, the immune checkpoint protein is selected from: PD-1, PD-L1, CTLA-4, LAG-3, OX40, CD28, CD40, CD47, CD70, CD80, CD122, GTIR, A2AR, B7-H3(CD276), B7-H4, IDO, KIR, Tim-3 or 4-1BB(CD137).

In some embodiments, the immune checkpoint protein is PD-1. In some embodiments, the immune checkpoint protein is PD-L1.

In some embodiments, the antigen binding domain targeting the immune checkpoint protein PD-1 has HCDR1 of an amino acid sequence as shown in SEQ ID NO: 62, HCDR2 of an amino acid sequence as shown in SEQ ID NO: 63, HCDR3 of an amino acid sequence as shown in SEQ ID NO: 64, LCDR1 of an amino acid sequence as shown in SEQ ID NO: 65, LCDR2 of an amino acid sequence as shown in SEQ ID NO: 66, and LCDR3 of an amino acid sequence as shown in SEQ ID NO: 67. In some embodiments, the antigen binding domain targeting the immune checkpoint protein PD-1 has a heavy chain variable region (VH) as shown in SEQ ID NO: 109 and a light chain variable region (VL) as shown in SEQ ID NO: 110. In some embodiments, the antigen binding domain that binds to the immune checkpoint protein PD-1 has a nucleic acid sequence as shown in SEQ ID NO: 25 or an amino acid sequence as shown in SEQ ID NO: 26.

In some embodiments, the antigen binding domain targeting the immune checkpoint protein PD-L1 has HCDR1 of an amino acid sequence as shown in SEQ ID NO: 68, HCDR2 of an amino acid sequence as shown in SEQ ID NO: 69, HCDR3 of an amino acid sequence as shown in SEQ ID NO: 70, LCDR1 of an amino acid sequence as shown in SEQ ID NO: 71, LCDR2 of an amino acid sequence as shown in SEQ ID NO: 72, and LCDR3 of an amino acid sequence as shown in SEQ ID NO: 73. In some embodiments, the antigen binding domain targeting the immune checkpoint protein PD-1 has VH as shown in SEQ ID NO: 111 and VL as shown in SEQ ID NO: 112. In some embodiments, the antigen binding domain that binds to the immune checkpoint protein PD-L1 has a nucleic acid sequence as shown in SEQ ID NO: 27 or an amino acid sequence as shown in SEQ ID NO: 28.

In some embodiments, the disease-associated cell surface antigen is selected from tumor antigens. In some embodiments, the tumor antigen is selected from: epidermal growth factor receptor family (EGFR, HER2, HER3, and HER4), PD-1, PD-L1, CTLA-4, 4-1BB(CD137), OX40, CD28, CD40, CD47, CD70, CD80, CD122, GTIR, A2AR, B7-H3(CD276), B7-H4, IDO, KIR, Tim-3, NY-ESO-1, GPC3, CLL-1, BCMA, mucin family (MUC1, MUC2, MUC3A, MUC3B, MUC4, MUC5AC, MUC5B, MUC6, MUC7, MUC8, MUC12, MUC13, MUC15, MUC16, MUC17, MUC19, and MUC20), CD19, CD20, CD22, CD30, CD33, CD52, chemistry chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, and CXCR6), PSMA, CEA, HDAC6, EpCAM, Mesothelin, TERT, TLR, TLR9, TLR4, CD33, GITR, Survivin, CD123, TIGIT, TIM-3, CD73, fibroblast growth factor receptor (FGFR), vascular endothelial growth factor receptor (FLT1, KDR/Flk-1, and VEGFR-3), hepatocyte growth factor receptor (HGFR), nerve growth factor receptor (NGFR), insulin-like growth factor receptor (IGFR), platelet-derived growth factor receptor (PDGFR) or hormone receptor (melanocortin 1 receptor (MC1 R, and MSHR)), and the like.

In some embodiments, the tumor antigen is CD19. In some embodiments, the tumor antigen is GPC3. In some embodiments, the tumor antigen is PSMA. In some embodiments, the tumor antigen is MUC16. In some embodiments, the tumor antigen is FAP.

In some embodiments, the antigen binding domain targeting the tumor antigen CD19 has HCDR1 of an amino acid sequence as shown in SEQ ID NO: 86, HCDR2 of an amino acid sequence as shown in SEQ ID NO: 87, HCDR3 of an amino acid sequence as shown in SEQ ID NO: 88, LCDR1 of an amino acid sequence as shown in SEQ ID NO: 89, LCDR2 of an amino acid sequence as shown in SEQ ID NO: 90, and LCDR3 of an amino acid sequence as shown in SEQ ID NO: 91. In some embodiments, the antigen binding domain targeting the tumor antigen CD19 has VH as shown in SEQ ID NO: 113 and VL as shown in SEQ ID NO: 114. In some embodiments, the antigen binding domain targeting the tumor antigen CD19 has a nucleic acid sequence as shown in SEQ ID NO: 52 or an amino acid sequence as shown in SEQ ID NO: 53.

In some embodiments, the antigen binding domain targeting the tumor antigen GPC3 has HCDR1 of an amino acid sequence as shown in SEQ ID NO: 80, HCDR2 of an amino acid sequence as shown in SEQ ID NO: 81, HCDR3 of an amino acid sequence as shown in SEQ ID NO: 82, LCDR1 of an amino acid sequence as shown in SEQ ID NO: 83,LCDR2 of an amino acid sequence as shown in SEQ ID NO: 84, and LCDR3 of an amino acid sequence as shown in SEQ ID NO: 85. In some embodiments, the antigen binding domain targeting the tumor antigen GPC3 has VH as shown in SEQ ID NO: 105 and VL as shown in SEQ ID NO: 106. In some embodiments, the antigen binding domain targeting the tumor antigen GPC3 has a nucleic acid sequence as shown in SEQ ID NO: 23 or an amino acid sequence as shown in SEQ ID NO: 24. In some embodiments, the CAR targeting the tumor antigen GPC3 has a nucleic acid sequence as shown in SEQ ID NO: 41 or an amino acid sequence as shown in SEQ ID NO:42.

In some embodiments, the antigen binding domain targeting the tumor antigen PSMA has HCDR1 of an amino acid sequence as shown in SEQ ID NO: 74, HCDR2 of an amino acid sequence as shown in SEQ ID NO: 75, HCDR3 of an amino acid sequence as shown in SEQ ID NO: 76, LCDR1 of an amino acid sequence as shown in SEQ ID NO: 77,LCDR2 of an amino acid sequence as shown in SEQ ID NO: 78, and LCDR3 of an amino acid sequence as shown in SEQ ID NO: 79. In some embodiments, the antigen binding domain targeting the tumor antigen PSMA has VH as shown in SEQ ID NO: 107 and VL as shown in SEQ ID NO: 108. In some embodiments, the antigen binding domain targeting the tumor antigen PSMA has a nucleic acid sequence as shown in SEQ ID NO: 32 or an amino acid sequence as shown in SEQ ID NO: 33. In some embodiments, the CAR targeting the tumor antigen GPC3 has the nucleic acid sequence as shown in SEQ ID NO: 32 or the amino acid sequence as shown in SEQ ID NO: 33.

In some embodiments, the antigen binding domain targeting the tumor antigen MUC16 has HCDR1 of an amino acid sequence as shown in SEQ ID NO: 93, HCDR2 of an amino acid sequence as shown in SEQ ID NO: 94, HCDR3 of an amino acid sequence as shown in SEQ ID NO: 95, LCDR1 of an amino acid sequence as shown in SEQ ID NO: 96, LCDR2 of an amino acid sequence as shown in SEQ ID NO: 97, and LCDR3 of an amino acid sequence as shown in SEQ ID NO: 98. In some embodiments, the antigen binding domain targeting the tumor antigen MUC16 has VH as shown in SEQ ID NO: 115 and VL as shown in SEQ ID NO: 116. In some embodiments, the antigen binding domain targeting the tumor antigen MUC16 has a nucleic acid sequence as shown in SEQ ID NO: 56 or an amino acid sequence as shown in SEQ ID NO: 57.

In some embodiments, the antigen binding domain targeting the tumor antigen FAP has HCDR1 of an amino acid sequence as shown in SEQ ID NO: 99, HCDR2 of an amino acid sequence as shown in SEQ ID NO: 100, HCDR3 of an amino acid sequence as shown in SEQ ID NO: 101, LCDR1 of an amino acid sequence as shown in SEQ ID NO: 102, LCDR2 of an amino acid sequence as shown in SEQ ID NO: 103, and LCDR3 of an amino acid sequence as shown in SEQ ID NO: 104. In some embodiments, the antigen binding domain targeting the tumor antigen FAP has VH as shown in SEQ ID NO: 117 and VL as shown in SEQ ID NO: 118. In some embodiments, the antigen binding domain targeting the tumor antigen FAP has a nucleic acid sequence as shown in SEQ ID NO: 58 or an amino acid sequence as shown in SEQ ID NO: 59.

In some embodiments, the antigen binding domain targeting the tumor antigen EGFR has HCDR1 of an amino acid sequence as shown in SEQ ID NO: 133, HCDR2 of an amino acid sequence as shown in SEQ ID NO: 134, HCDR3 of an amino acid sequence as shown in SEQ ID NO: 135, LCDR1 of an amino acid sequence as shown in SEQ ID NO: 136, LCDR2 of an amino acid sequence as shown in SEQ ID NO: 137, and LCDR3 of an amino acid sequence as shown in SEQ ID NO: 138. In some embodiments, the antigen binding domain targeting the tumor antigen EGFR has VH as shown in SEQ ID NO: 119 and VL as shown in SEQ ID NO: 120.

In some embodiments, the antigen binding domain targeting the tumor antigen HER2 has HCDR1 of an amino acid sequence as shown in SEQ ID NO: 139, HCDR2 of an amino acid sequence as shown in SEQ ID NO: 140, HCDR3 of an amino acid sequence as shown in SEQ ID NO: 141, LCDR1 of an amino acid sequence as shown in SEQ ID NO: 142, LCDR2 of an amino acid sequence as shown in SEQ ID NO: 143, and LCDR3 of an amino acid sequence as shown in SEQ ID NO: 144. In some embodiments, the antigen binding domain targeting the tumor antigen HER2 has VH as shown in SEQ ID NO: 121 and VL as shown in SEQ ID NO: 122.

In some embodiments, the antigen binding domain targeting the tumor antigen CD3 has HCDR1 of an amino acid sequence as shown in SEQ ID NO: 127, HCDR2 of an amino acid sequence as shown in SEQ ID NO: 128, HCDR3 of an amino acid sequence as shown in SEQ ID NO: 129, LCDR1 of an amino acid sequence as shown in SEQ ID NO: 130, LCDR2 of an amino acid sequence as shown in SEQ ID NO: 131, and LCDR3 of an amino acid sequence as shown in SEQ ID NO: 132. In some embodiments, the antigen binding domain targeting the tumor antigen CD3 has VH as shown in SEQ ID NO: 123 and VL as shown in SEQ ID NO: 124.

In some embodiments, the CAR-modified T cell of the present disclosure further express a second CAR, wherein the second CAR includes an antigen binding domain and a transmembrane domain.

In some embodiments, the antigen binding domain of the second CAR binds to a disease-associated cell surface antigen. In some embodiments, the disease-associated cell surface antigen is a tumor antigen. In some embodiments, the tumor antigen is selected from: epidermal growth factor receptor family (EGFR, HER2, HER3, and HER4), PD-1, PD-L1, CTLA-4, 4-1BB(CD137), OX40, CD28, CD40, CD47, CD70, CD80, CD122, GTIR, A2AR, B7-H3(CD276), B7-H4, IDO, KIR, Tim-3, NY-ESO-1, GPC3, CLL-1, BCMA, mucin family (MUC1, MUC2, MUC3A, MUC3B, MUC4, MUC5AC, MUCSB, MUC6, MUC7, MUC8, MUC12, MUC13, MUC15, MUC16, MUC17, MUC19, and MUC20), CD19, CD20, CD22, CD30, CD33, CD52, chemistry chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, and CXCR6), PSMA, CEA, HDAC6, EpCAM, Mesothelin, TERT, TLR, TLR9, TLR4, CD33, GITR, Survivin, CD123, TIGIT, TIM-3, CD73, FGFR, vascular endothelial growth factor receptor (FLT1, KDR/Flk-1, and VEGFR-3), HGFR, NGFR, IGFR, PDGFR or hormone receptor (melanocortin 1 receptor (MC1R, and MSHR)). In some embodiments, the chimeric antigen receptor 2 binds to the tumor antigen EGFR.

In some embodiments, the CAR-modified T cells of the present disclosure further express secretory polypeptides selected from but not limited to: one or two of immune function modulatory factors, antibodies specifically targeting the tumor antigens, and antibodies specifically targeting the immune checkpoints.

In some embodiments, the secretory polypeptide is an immune function regulatory factor. In some embodiments, the immune function regulatory factor is selected from IL-2, IL-12, IL-7, IL-15, IL-18, IL-21, IL-24, 4-1BBL, PD-1 extracellular region, PD-L1 extracellular region, CCL19, MIP-1α, GM-CSF, IFN-α, IFN-β, IFN-γ, TNF-γ, M-CSF, TGF-β or TRAIL, etc.

In some embodiments, the secretory polypeptide is an immune function regulatory factor IL-2. In some embodiments, the secretory polypeptide is an immune function regulatory factor IL-12. In some embodiments, the secretory polypeptide is an immune function regulatory factor IL-15. In some embodiments, the secretory polypeptide is an immune function regulatory factor IL-18. In some embodiments, the secretory polypeptide is an immune function regulatory factor PD-1 mutant polypeptide. In some embodiments, the immune function regulatory factor IL-2 has a nucleic acid sequence as shown in SEQ ID NO: 15 or an amino acid sequence as shown in SEQ ID NO: 16. In some embodiments, the immune function regulatory factor IL-12 has a nucleic acid sequence as shown in SEQ ID NO: 17 or an amino acid sequence as shown in SEQ ID NO: 18. In some embodiments, the immune function regulatory factor IL-15 has a nucleic acid sequence as shown in SEQ ID NO: 19 or an amino acid sequence as shown in SEQ ID NO: 20. In some embodiments, the immune function regulatory factor IL-2 has a nucleic acid sequence as shown in SEQ ID NO: 21 or an amino acid sequence as shown in SEQ ID NO:22.

In some embodiments, the secretory polypeptide is an antibody specifically targeting a tumor antigen. In some embodiments, the tumor antigen is selected from: epidermal growth factor receptor family (EGFR, HER2, HER3, and HER4), PD-1, PD-L1, CTLA-4, 4-1BB(CD137), OX40, CD28, CD40, CD47, CD70, CD80, CD122, GTIR, A2AR, B7-H3(CD276), B7-H4, IDO, KIR, Tim-3, NY-ESO-1, GPC3, CLL-1, BCMA, mucin family (MUC1, MUC2, MUC3A, MUC3B, MUC4, MUC5AC, MUC5B, MUC6, MUC7, MUC8, MUC12, MUC13, MUC15, MUC16, MUC17, MUC19, and MUC20), CD19, CD20, CD22, CD30, CD33, CD52, chemistry chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, and CXCR6), PSMA, CEA, HDAC6, EpCAM, Mesothelin, TERT, TLR, TLR9, TLR4, CD33, GITR, Survivin, CD123, TIGIT, TIM-3, CD73, FGFR, vascular endothelial growth factor receptor (FLT1, KDR/Flk-1, and VEGFR-3), HGFR, NGFR, IGFR, PDGFR or hormone receptor (melanocortin 1 receptor (MC1R, and MSHR)) and the like. In some embodiments, the immune checkpoint protein is selected from: PD-1, PD-L1, CTLA-4, LAG-3, OX40, CD28, CD40, CD47, CD70, CD80, CD122, GTIR, A2AR, B7-H3(CD276), B7-H4, IDO, KIR, Tim-3, or 4-1BB(CD137). In some embodiments, the secretory polypeptide is a bispecific antibody. In some embodiments, the secretory polypeptide is a bispecific antibody targeting FAP and CD3. In some embodiments, the secretory polypeptide targeting FAP and CD3 has an amino acid sequence as shown in SEQ ID NO: 125. In some embodiments, the secretory polypeptide is a bispecific antibody targeting HER2 and CD3. In some embodiments, the secretory polypeptide targeting HER2 and CD3 has an amino acid sequence as shown in SEQ ID NO: 126. In some embodiments, the secretory polypeptide is a bispecific antibody targeting CD19 and CD3.

In some embodiments, the secretory polypeptide is an antibody specifically targeting an immune checkpoint. In some embodiments, the immune checkpoint protein is selected from: PD-1, PD-L1, CTLA-4, LAG-3, OX40, CD28, CD40, CD47, CD70, CD80, CD122, GTIR, A2AR, B7-H3(CD276), B7-H4, IDO, KIR, Tim-3 or 4-1BB(CD137). In some embodiments, the immune checkpoint protein is PD-1. In some embodiments, the immune checkpoint protein is PD-L1. In some embodiments, the antibody targeting the immune checkpoint protein PD-1 has a nucleic acid sequence as shown in SEQ ID NO: 25 or an amino acid sequence as shown in SEQ ID NO: 26. In some embodiments, the antigen binding domain that binds to the immune checkpoint protein PD-L1 has a nucleic acid sequence as shown in SEQ ID NO: 27 or an amino acid sequence as shown in SEQ ID NO: 28.

In some embodiments, the secretory polypeptide is constitutively expressed. In some embodiments, the constitutively expressed secretory polypeptide is on polypeptide chain of the first or second CAR. In some embodiments, the constitutively expressed secretory polypeptide is linked to the first or second CAR by a self-cleaving peptide. In some embodiments, the self-cleaving peptide is selected from P2A or T2A. In some embodiments, the self-cleaving peptide P2A has a nucleic acid sequence as shown in SEQ ID NO: 29 or an amino acid sequence as shown in SEQ ID NO: 30.

In some embodiments, the first CAR targets a tumor antigen, and the constitutively expressed secretory polypeptide is an immune function regulatory factor. In some embodiments, the first CAR targets the tumor antigen PSMA, and the constitutively expressed secretory polypeptide is immune function regulatory factor IL-12. In some embodiments, the CAR targeting PSMA and constitutively expressing IL-12 has a nucleic acid sequence as shown in SEQ ID NO: 40.

In some embodiments, the first CAR targets a tumor antigen, and the constitutively expressed secretory polypeptide is an antibody specifically targeting an immune checkpoint. In some embodiments, the first CAR targets tumor antigen GPC3, and the constitutively expressed secretory polypeptide is an antibody specifically targeting immune checkpoint PD-1. In some embodiments, the CAR targeting GPC3 and expressing the PD-1 antibody has a nucleic acid sequence as shown in SEQ ID NO: 47. In some embodiments, the CAR targeting GPC3 and expressing the PD-1 antibody has a nucleic acid sequence as shown in SEQ ID NO: 48.

In some embodiments, the first CAR targets a tumor antigen, and the constitutively expressed secretory polypeptides include an immune function regulatory factor and an antibody specifically targeting an immune checkpoint protein. In some embodiments, the first CAR targets tumor antigen GPC3, and the constitutively expressed secretory polypeptide includes immune function regulatory factor IL-12 and an antibody specifically targeting immune checkpoint PD-1. In some embodiments, the CAR targeting GPC3 and expressing IL-12 and specifically targeting the PD-1 antibody has a nucleic acid sequence as shown in SEQ ID NO: 50.

In some embodiments, the secretory polypeptide is inducibly expressed. In some embodiments, the inducibly secretorypolypeptide is expressed after activation by an inducible promoter. In some embodiments, the inducible promoter is 6XNFAT. In some embodiments, the 6XNFAT promoter has a sequence as shown in SEQ ID NO. 3. In some embodiments, the inducible promoter is NF-κb responsive promoter. Further, the NF-κb responsive promoter includes an NF-κb response element and a basal promoter. In some embodiments, the NF-κb response element has a sequence as shown in one of SEQ ID NO. 149 to SEQ ID NO. 152. In some embodiments, the NF-κb basal promoter has a sequence as shown in one of SEQ ID NO. 153 to SEQ ID NO. 155. In some embodiments, the NF-κb responsive promoter has a sequence as shown in SEQ ID NO. 156.

In some embodiments, the secretory polypeptide is on a different polypeptide chain from the first or second CAR. In some embodiments, the secretory polypeptide is on the first or second CAR polypeptide. In some embodiments, the secretory polypeptide is linked to the CAR by a self-cleaving peptide. In some embodiments, the self-cleaving peptide is selected from P2A or T2A. In some embodiments, the self-cleaving peptide P2A has a nucleic acid sequence as shown in SEQ ID NO: 29 or an amino acid sequence as shown in SEQ ID NO: 30. In some embodiments, the coding sequence of the inducibly expressed secretorypolypeptide and the first or second CAR are linked by a promoter and a signal peptide coding sequence.

In some embodiments, the first CAR targets a tumor antigen, and the inducibly expressed secretorypolypeptide is an immune function regulatory factor.

In some embodiments, the first CAR targets tumor antigen PSMA, and the inducibly expressed secretory polypeptide is immune function regulatory factor IL-2. In some embodiments, the first CAR targeting PSMA with inducibly expressed IL-2 has a nucleic acid sequence as shown in SEQ ID NO: 39. In some embodiments, the first CAR targets tumor antigen PSMA, and the inducibly expressed secretory polypeptide is immune function regulatory factor IL-12. In some embodiments, the CAR targeting PSMA with inducibly expressed IL-12 has a nucleic acid sequence as shown in SEQ ID NO: 34. In some embodiments, the first CAR targets tumor antigen PSMA, and the inducibly expressed secretory polypeptide is immune function regulatory factor IL-15. In some embodiments, the CAR targeting PSMA within ducibly expressed IL-15 has a nucleic acid sequence as shown in SEQ ID NO: 36.In some embodiments, the first CAR targets tumor antigen PSMA, and the inducibly expressed secretory polypeptide is immune function regulatory factor IL-18. In some embodiments, the CAR targeting PSMA with inducibly expressed IL-18 has a nucleic acid sequence as shown in SEQ ID NO: 36. In some embodiments, the first CAR targets tumor antigen GPC3, and the inducibly expressed secretory polypeptide is immune function regulatory factor IL-2. In some embodiments, the CAR targeting PSMA with inducibly expressed IL-2 has a nucleic acid sequence as shown in SEQ ID NO: 49.

In some embodiments, the first CAR targets a tumor antigen, and the inducibly expressed secretory polypeptide is an antibody that specifically binds to an immune checkpoint protein.

In some embodiments, the first CAR targets tumor antigen GPC3, and the inducibly expressed secretorypolypeptide is an antibody that specifically binds to immune checkpoint protein PD-1. In some embodiments, the CAR targeting GPC3 with inducibly expressed the PD-1 antibody has a nucleic acid sequence as shown in SEQ ID NO: 43. In some embodiments, the first CAR targets tumor antigen GPC3, and the inducibly expressed secretory polypeptide is an antibody that specifically binds to immune checkpoint protein PD-L1. In some embodiments, the CAR targeting GPC3 with inducibly expressed the PD-L1 antibody has a nucleic acid sequence as shown in SEQ ID NO: 45.

In some embodiments, the first CAR targets a tumor antigen, and the inducibly expressed secretory polypeptide includes an immune function regulatory factor and an antibody that specifically binds to an immune checkpoint protein.

In some embodiments, the first CAR targets tumor antigen GPC3, and the inducibly expressed secretory polypeptide includes immune function regulatory factor IL-12 and an antibody that specifically targets immune checkpoint PD-1. In some embodiments, the CAR targeting GPC3 with inducibly express IL-2 and PD-1 antibodies has a nucleic acid sequence as shown in SEQ ID NO: 51.

In some embodiments, CAR containing costimulatory signal domains (such as a 4-1BB intracellular costimulatory signal, intracellular signal conduction domains of 4-1BB and OX40, intracellular costimulatory signal conduction domains of 4-1BB and CD28, intracellular signal conduction domains of 4-1BB and LAG3, or intracellular signal conduction domains of 4-1BB, CD28 and OX40), tumor recognition molecule aPSMA scfv, immune function regulatory factor (IL-2, IL-12, IL-15 or IL-18) but without the first signal is artificially introduced into tumor infiltrating lymphocyte (TIL). After entering tumor tissue by the specificity of aPSMA scfv and the tissue specificity of the natural TCR-CD3 complex on the cell surface, CART cells are activated by the first signal provided by the combination of TCR-CD3 complex and antigen peptide-MHC-I molecule, and their anti-tumor effects are enhanced by the secretory immune regulatory factors (such as IL-2, IL-12, IL-15 or IL-18). Tumor recognition and binding by aPSMA scfv as well as the activation of intracellular signal conduction of the engineered costimulatory signal molecule maintain the survival of the activated CART cells.

In some embodiments, CAR containing costimulatory signal domain (such as a 4-1BB intracellular costimulatory signal domain, intracellular signal conduction domains of 4-1BB and OX40, intracellular costimulatory signal conduction domains of 4-1BB and CD28, intracellular signal conduction domains of 4-1BB and LAG3, or intracellular signal conduction domains of 4-1BB, CD28 and OX40), tumor recognition molecule aGPC3 scfv, immune function regulatory factor (IL-12, IL-15, or IL-18) and/or scfvs targeting an immune checkpoint protein but without the first signal is artificially introduced into TIL. After entering the tumor tissue by the specificity of aGPC3 scfv on its CAR and the tissue specificity of the natural TCR-CD3 complex on the cell surface, CART cells are activated by the first signal provided by the combination of TCR-CD3 complex and antigen peptide-MHC-I molecule, and their anti-tumor effects are enhanced by the secretory immune regulatory factors (such as IL-12, IL-15 or IL-18), or by the breakdown of the immune tolerance microenvironment of the tumor tissue by the secretory scfv targeting the immune checkpoint protein (such as PD-1 or PD-L1). Tumor recognition and binding by the tumor cell recognition molecule aGPC3 scfv as well as the activation of intracellular signal conduction of the engineered costimulatory signal molecule maintain the survival of the activated CART cells.

### BRIEF DESCRIPTION OF THE DRAWINGS

Drawings that constitute the present application are used to provide further understanding of the present disclosure. The exemplary embodiments and descriptions of the present disclosure thereof are used to explain the present disclosure, and do not constitute improper limitation to the present disclosure. In the drawings:

FIG. 1 shows expression of CAR on the surface of CART cells targeting PSMA.

FIG. 2A-2J show expression of CAR on the surface of CARTs cell targeting GPC-3.

FIG. 3A-3D shows levels of IL-2, IFN-γ, IL-12 and IL-18 secretory by PSMA-targeted CART effector cells after co-incubation with target cells PC3, LnCAP or 22RV1 at a ratio of 1:1. Herein Mock is PBMC cells without genetic modification, 1 is aPSMA BBZ, 2 is aPSMA BBZ+iIL-12, 3 is aPSMA BBZ+iIL-15, 4 is aPSMA BB+iIL-2, 5 is aPSMA BB+iIL-18, and 6 is aPSMA BB+IL-12.

FIG. 4 shows the level of IL-2 or aPD-1 scfv secretory by GPC-3-targeted CART cells after co-incubation with target cells A431 or HepG2 at a ratio of 1:1. In FIG. 4A, Mock is PBMC cells without genetic modification, 1 is aGPC-3BBZ, 2 is aGPC-3 BB+iaPD-1, 3 is aGPC-3 BB+iaPD-L1, 4 is aGPC-3 BB-PGK+aPD-1, 5 is aGPC-3 BB-P2A+iaPD-1, and 6 is aGPC-3 BB+iIL-2; and in FIG. 4B, Mock is PBMC cells without genetic modification, 1 is aGPC-3BBZ, 2 is aGPC-3 BB+iaPD1, 3 is aGPC-3 BBZ+iaPD-1, 4 is aGPC-3 BB-P2A+aPD-1, 5 is aGPC-3 Z+iaPD-1, 6 is aGPC-3 BB+iIL-2, 7 is aGPC-3 Z+iIL-2, and 8 is positive control nivolumab. 5 and 7 are CD3Z only CAR.

FIG. 5 shows killing activity of PMSA-targeted CART cells after co-incubation with target cells LNCaP or PC3 at different ratios, herein Mock is PBMC cells without genetic modification.

FIG. 6 shows killing activity of GPC-3-targeted CART cells after co-incubation with a target cell HepG2 or A431 at different ratios, herein Mock is PBMC cells without genetic modification.

FIG. 7 is a schematic diagram of CAR.

FIG. 8 shows the killing activity of CAR-T cells containing only costimulatory signal transduction domain with the first activation signal mediated by tumor-specific BiTE.

FIG. 9 shows the killing effect of patient derived CART cells expressing aPSMA BBZ towards tumor cells in vitro.

FIG. 10 shows the activation of NF-κb responsive promoter by TNFα, Phorbol myristate acetate (PMA) and 4-1bbL.

FIG. 11 shows that CART cells expressing aPSMA BBZ could effectively inhibit the growth of a PSMA-positive solid tumors in mice.

### DETAILED DESCRIPTION OF THE EMBODIMENTS

The present disclosure is described in detail herein by reference to the following definitions and embodiments. The contents of all patents and publications mentioned herein, including all sequences disclosed in these patents and publications, are expressly incorporated herein by reference.

The term “chimeric antigen receptor” or the term “CAR” is an artificially engineered receptor. From extracellular to intracellular, the “CAR” of the present disclosure includes: a) antigen binding domain; b) transmembrane domain; and c) intracellular costimulatory signal transduction domain., “CAR” of the present disclosure includes an artificially introduced costimulatory signal transduction domain, and does not contain an artificially introduced first signal transduction domain. The “chimeric antigen receptor-modified T (CART) cells” or “CAR-modified T cells” of the present disclosure have the following characteristics: the CART cells contain a natural, unmodified first signal transduction domain CD3zeta, and an artificially introduced costimulatory signal transduction domain, wherein the natural, unmodified first signal is provided by the natural TCR-CD3 complex on the surface of the CART cells.

The term “first signal” refers to T cell activation signal provided by the binding of the TCR-CD3 complex on the surface of the T cells to antigenic peptide-MHC molecules. Different from the first signal of traditional CART cells (the first to the fourth generation) provided by CD3zeta on CAR and TCR on T cells, the “first signal” of the chimeric antigen receptor-modified T cells of the present disclosure is provided by the natural TCR-CD3 complex on the surface of T cells.

The term “costimulatory signal” refers to the second signal provided by binding of costimulatory molecules (such as CD28) on the surface of T cell to its ligand (such as B7). The “second signal” of the chimeric antigen receptor-modified T cell of the present disclosure is provided by the artificially introduced costimulatory signal transduction domain on CAR.

The term “signal transduction domain” refers to a functional portion of a protein, and the functional portion exerts the following functions by transmitting information inside the cell: regulating cell activities by generating a second messenger or as an effector in response to such messengers.

The term “first signal transduction domain” refers to a signal transduction domain that regulates the primary activation of the TCR-CD3 complex in a stimulatory manner or in an inhibitory manner. The primary signal transduction domain that acts in the stimulatory manner may contain a signal transduction motif called immunoreceptor tyrosine-based activation motif (ITAM). Illustrative examples of ITAM containing the first signal transduction domain include, but are not limited to, those derived from FcRγ, FcRβ, CD3γ, CD3ε, CD3δ, CD3zeta, CD22, CD79a, CD79b, and CD66d.

The term “intracellular signal transduction domain of costimulatory protein” may be the intracellular portion of a costimulatory protein, which may contain all the intracellular part or all the natural intracellular signal transduction domain of, or its functional fragment or derivative thereof. “Costimulatory protein” refers to some adhesion molecules on the surface of immune cells (such as T cells). These adhesion molecules bind to their ligands to activate the immune cells (such as T cells), thus enhancing the proliferation and cytokine secretion as well as persistence of the activated immune cells. “costimulatory proteins” include, but are not limited to: MHC class-I molecule, BTLA and Toll ligand receptor, and OX40, CD27, CD28, CDS, ICAM-1, LFA-1(CD11a/CD18), ICOS(CD278), 4-1BB(CD137), GITR, BAFFR, HVEM(LIGHTR), SLAMF7, NKp80(KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1 , ITGB7, NKG2D, NKG2C, TNFR2, TRANCE/RANKL, DNAM1(CD226), SLAMF4(CD244, 2B4), CD84, CD96(Tactile), CEACAM1, CRTAM, Ly9(CD229), CD160(BY55), PSGL1, CD100(SEMA4D), CD69, SLAMF6(NTB-A, Ly108), SLAM (SLAMF1, CD150, IPO-3), BLAME (SLAMF8), SELPLG (CD162), LTBR, LAT, GADS, SLP-76, PAG/Cbp, CD19a and ligand that specifically binds to CD83.

The term “antigen binding domain” may have any structures as long as it binds to targetantigen. The domain may include, for example, variable regions of antibody heavy and light chains (for example, 1 to 6 CDRs); a module about 35 amino acids called domain A, which is contained in Avimer (a cell membrane protein existing in vivo) (WO2004/044011 and WO2005/040229); 10Fn3 domain-containing Adnectin that binds to a protein in glycoprotein fibronectin expressed on the cell membrane (WO2002/032925); Affibody which has an IgG-binding scaffold(a triple-helix of 58 amino acids which constituting the protein A) (WO1995/001937); the designed Ankyrin repeat proteins (DARPins), which are exposed on the molecular surface of an ankyrin repeat (AR), wherein the AR has such a structure with repeat stack of subunits containing aa turn with 33 amino acid residues, two antiparallel helix, and a loop (WO2002/020565); Anticalins and the like, which are tetracyclic regions at one side of a centrally twisted barrel structure of eight antiparallel chains that are highly conserved in supporting molecule neutrophil gelatinase-associated lipocalin (NGAL) (WO2003/029462) and concave regions formed by a parallel sheet structure inside a horseshoe-shaped structure formed by repeated stacking of a leucine-rich repeat (LRR) module of a variable lymphocyte receptor (VLR) (it does not have an immunoglobulin structure and is the system for acquired immunity in a jawless vertebrate such as lampreys and hagfish) (WO2008/016854). As a preferred example of the antigen binding domain of the present disclosure, it may be an antigen binding domain containing variable regions of heavy and light chains of an antibody. As an example of such an antigen binding domain, it may be “scFv (single-chain Fv)”, “single-chain antibody”, “Fv”, “scFv2 (single-chain Fv2)”, “Fab” or “F(ab')2” and the like.

The term “antibody” is used in the broadest sense as long as it exerts the desired biological activity, wherein it may include monoclonal antibodies (including full-length monoclonal antibodies), polyclonal antibodies, antibody variants, antibody fragments (including but not limited to Fab, Fab′, F(ab′)2, Fv fragment, scFv fragment, disulfide-linked Fvs (sdFv), Fd fragments consisting of VH and CH1 domain, linear antibodies, single domain antibodies such as sdAb (VL or VH), camelid VHH domains, multispecific antibodiesor isolated CDRs or other epitope binding fragments formed by antibody fragments (for example, a bivalent fragment including two Fab fragments linked by disulfide bonds at the hinge region), multispecific antibodies (multiple specific antibodies) (for example, bispecific antibodies (dual specific antibodies), chimeric antibodies, humanized antibodies and other antibodies.

“Disease-associated cell surface antigen” of the present disclosure refers to cell surface antigens that are associated with disease treatment and diagnosis, expressed on the cell surface, and may be targeted by CAR. The “disease-associated cell surface antigen” of the present disclosure includes CD antigens, immune checkpoint antigen, tumor-associated antigens, tumor-specific antigens, virus-induced tumor antigens, and may be selected from but not limited to the following antigens: CD19, PD-1, PD-L1, HER2, STAT3, CTLA-4, IDO, NY-ESO-1, CD40, CSF1R, BCMA, MUC1, ADORA2A, CD20, GD2, TLR7, VVT1, IFNAR1, CD47, Neoantigen, EGFR, LAG- 3. OX40, PSMA, Mesothelin, TERT, TLR, TLR9, 4-1BB, IL2R, TLR4, CD33, GITR, HPV E6, Survivin, CD123, TIGITTIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STATS, TGFB1, TLR2, USP7, ANG1, ANG2, B7-H3, CLEC12A, IL13RA2, RIG-I, TRP2, VEGF, AFP, Alpha-Gal, COX-2, EPHA2, gp96, MUC16, p53, TGF-β, CD138, CDw136, CS1, CXCR2, EGFRvIII, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, ASPH, AXL, BCL2, BTE6-LX-8b, BTE6-X-15-7, Carbohydrate antigens, CCL20, CCL3, CCNB1, CD147, CD155, CD16, CD162, CD16a, CD200, CD21, CD28 , CD44, CD52, CD54, CD7, CD80, CD88, Claudin 18, cMET, COX2, CSF1, CTCFL, CXCR5, CXCR7, E1A, EIF2AK3, ERG, FGF2, FN1, GC, GM2, gpA33, HBV, Hemagglutinin, HER3, HILPDA, HLA-DR, HMW-MAA, HP59, HPV 16, HPV E617, HPV L1, HSP105, HSP65, HVEM, Hyaluronan, IL13RA1, IL2, IL21R, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, 5′Nucleotidase (Ecto 5′Nucleotidase or CD73 or NT5E or EC 3.1.3.5), ADAM9, AIM2, B7-H6, BAFF-R, BAI1, BARD1, BOB-1, CA9, Cancer testis antigen, CB2, CBLB, CC R9, CD13, CD130, CD150, CD160, CD200R1, CD267, CD29, CD3E, CD4, CD51, CD8, CGEN-XXXX, Claudin 6, CLEC2D, COX, COX-1, CPEB4, CPEG4, CRBN, CRLF2, CSPG4, CTA, CXCL1, CXCR3, Cytosine deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GaINAc, GARP, Gelactin-9, Gelatcin-1/3/9, GLD18, GNRHR, GP160, GP73, H3.3K27M, HAGE, HDAC2, HDAC8, HPV16E6, HPV16E7, HSP, Hypoxia, ICAM, ICAM7, IDO1, IFNG, IFNGR2, IGF2R, IGFBP2, IGK, IL10RA, IL12RB1, IL13, IL13R, IL13Ralpha2, IL15RA, IL17A, IL17B, IL1A, IL1R1, IL1R3, IL21, IL22R, IL27R, IL2RA, IL35, IL9R, Integrin beta-7, IRAK1, ITGB5, Kappa myeloma antigen, KIR2DL2, Kynurenine, L1CAM, Lambda Myeloma Antigen, LAMP, LLO, LXRA, LXRB, Mas receptor, MG7, MHCI, MHCII, MIC, MOSPD2, MRP-3, MRP1, MRP3765, muGNTP01, MYB, MYBL2, NFAT, NGcGM3, Nrf2, p38MAP Kinase (EC 2.7.11.24), P55, PAM4, PAP, PASD1, PCDH18, PD-L2, PI3K-delta, POTE, PPT, Protein tolemerase, PTGER2, RANKL, RBL001, RNF43, ROR2, S100A9, SEREX, SLAMF1, STAT, TACSTD2, TASTD2, TDO2, TEM, thymidine kinase, Thymidylate synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, ZP3.

“Immune function regulatory/modulatory factor” refers to any cytokine or molecule that can regulate the process or outcome of an immune response. The “immune function regulatory factor” includes immune function stimulating factors and immune function inhibiting factors. The “immune function regulatory factor” includes but is not limited to: IL-2, IL-12, IL-7, IL-15, IL-18, IL-21, IL-24, 4-1BBL, PD-1 extracellular region, PD-L1 extracellular region, CCL19, MIP-1a, GM-CSF, IFN-α, IFN-β, IFN-γ, TNF-α, M-CSF, TGF-β and TRAIL and the like.

“Expression vector” of the present disclosure refers to a vector containing a recombinant polynucleotide which contains an expression control sequence operably linked to a coding nucleotide sequence. The expression vector includes sufficient cis-acting element for expression, and other expression elements may be provided by host cells or in an in vitro expression system. The expression vector includes all those known in the art, including cosmids, plasmids (for example, naked or contained in a liposome) and viruses (for example, lentivirus, retrovirus, adenovirus and adeno-associated virus) that are incorporated with the recombinant polynucleotide.

The term “lentiviral vector” refers to a vector derived from at least a part of the lentiviral genome, and specifically includes a self-inactivating lentiviral vector as provided in Milone et al., Mol. Ther. 17(8): 1453-1464 (2009). Other examples of the lentiviral vector that may be used in clinical practice include, but are not limited to Lentivector® gene delivery technology from Oxford BioMedica, and LLENTIMAX vector system from Lentigen. Non-clinical types of lentiviral vectors may also available and may be known to those skilled in the art.

“Constitutive” expression includes the state in which a gene product is produced in a living cell under most or all physiological conditions of the cell.

### EMBODIMENT

It should be noted that, in the case without conflicting, the embodiments in the present application are merely illustrative, and are not intended to limit the present disclosure in any manner.

### Embodiment

### Embodiment 1: Construction of Recombinant Lentiviral Vector of Chimeric Antigen Receptor

Recombinant vectors containing the following sequences are constructed (expression framework from 5-end to 3-end):

1) Vector Targeting PSMA:

EF1a promoter-CD8α signal peptide-aPSMA scfv-CD8 hinge-CD8 transmembrane region-4-1BB (aPSMA BB)

EF1a promoter-CD8α signal peptide-aPSMA scfv-CD8 hinge-CD8 transmembrane region-4-1BB-CD3Z (aPSMA BBZ)

2) Vector Targeting PSMA and Inducibly Secreting IL-12:

EF1a promoter-CD8α signal peptide-aPSMA scfv-CD8 hinge-CD8 transmembrane region-4-1BB-6XNFAT promoter-IgG kappa signal peptide-IL-12 (P40-35) (aPSMA BB+iIL-12)

EF1a promoter-CD8α signal peptide-aPSMA scfv-CD8 hinge-CD8 transmembrane region-4-1BB-CD3Z-6XNFAT promoter-IgG kappa signal peptide-IL-12(P40-35) (aPSMA BBZ+iIL-12)

3) Vector Targeting PSMA and Inducibly Secreting IL-12:

EF1a promoter-CD8α signal peptide-aPSMA scfv-CD8 hinge-CD8 transmembrane region-4-1BB-6XNFAT promoter-IgG kappa signal peptide-IL-15 (aPSMA BB+iIL-15)

EF1a promoter-CD8α signal peptide-aPSMA scfv-CD8 hinge-CD8 transmembrane region-4-1BB-CD3Z-6XNFAT promoter-IgG kappa signal peptide-IL-15 (aPSMA BBZ+iIL-15)

4) Vector Targeting PSMA and Inducibly Secreting IL-18:

EF1a promoter-CD8α signal peptide-aPSMA BB-CD8 hinge-CD8 transmembrane region-4-1BB-6XNFAT promoter-IgG kappa signal peptide-IL-18 (aPSMA BB+iIL-18)

5) Vector Targeting PSMA and Inducibly Secreting IL-2:

EF1a promoter-CD8α signal peptide-aPSMA scfv-CD8 hinge-CD8 transmembrane region-4-1BB-6XNFAT promoter-IgG kappa signal peptide-IL-2 (aPSMA BB+iIL-2)

6) Vector Targeting PSMA and Constitutively Secreting IL-12:

EF1a promoter-CD8a signal peptide-aPSMA scfv-CD8 hinge-CD8 transmembrane region-4-1BB-PGK promoter-IgG kappa signal peptide-IL-12 (P40-35) (aPSMA BB-PGK+IL-12)

7) Vector Targeting GPC3:

EF1a promoter-CD8α signal peptide-aGPC3 scfv-CD8 hinge-CD8 transmembrane region-4-1BB (aGPC-3BB)

EF1a promoter-CD8α signal peptide-aGPC3 scfv-CD8 hinge-CD8 transmembrane region-4-1BB-CD3Z (aGPC-3BBZ)

8) Vector Targeting GPC3 and Inducibly Secreting aPD-1:

EF1 a promoter-CD8α signal peptide-aGPC3scfv-CD8 hinge-CD8 transmembrane region-4-1BB-6XNFAT promoter-IgG kappa signal peptide-aPD-1 scfv (aGPC-3BB+iaPD-1)

EF1a promoter-CD8α signal peptide-aGPC3 scfv-CD8 hinge-CD8 transmembrane region-4-1BB-CD3Z-6XNFAT promoter-IgG kappa signal peptide-aPD-1 scfv (aGPC-3BBZ+iaPD-1)

9) Vector Targeting GPC3 and Inducibly Secreting aPD-L1:

EF1a promoter-CD8α signal peptide-aGPC3scfv-CD8 hinge-CD8 transmembrane region-4-1BB-6XNFAT promoter-IgG kappa signal peptide-aPD-L1 scfv (aGPC-3BB+iaPD-L1)

EF1a promoter-CD8α signal peptide-aGPC3scfv-CD8 hinge-CD8 transmembrane region-4-1BB-CD3Z-6XNFAT promoter-IgG kappa signal peptide-aPD-L1 scfv (aGPC-3BBZ+iaPD-L1)

10) Vector Targeting GPC3 and Constitutively Secreting aPD-1:EF1a Promoter-CD8α Signal Peptide-aGPC3scfv-CD8hinge-CD8 Transmembrane Region-4-1BB-PGK Promoter-IgG Kappa Signal Peptide-aPD-1 scfv (aGPC-3BB-PGK+aPD-1)

11) Vector Targeting GPC3 and Constitutively Secreting aPD-1:

EF1a promoter-CD8α signal peptide-aGPC3scfv-CD8 hinge-CD8 transmembrane region-4-1BB-P2A-IgG kappa signal peptide-aPD-1 scfv (aGPC-3BB-P2A+aPD-1)

12) Vector Targeting GPC3 and Inducibly Secreting IL-2: EF1a Promoter-CD8α Signal Peptide-aGPC3scfv-CD8 Hinge-CD8 Transmembrane Region-4-1BB-6XNFAT Promoter-IgG Kappa Signal Peptide-iIL-2 (aGPC-3BB+iIL-2)

13) Vector Targeting GPC3 and Constitutively Secreting IL-12 (p40-35), and aPD-1:

EF1a promoter-CD8α signal peptide-aGPC3scfv-CD8 hinge-CD8 transmembrane region-4-1BB-PGK promoter-IgG kappa signal peptide-IL-12(p40-35)-P2A-IgG kappa signal peptide-aPD-1 scfv (aGPC-3BB-PGK+IL-12-P2A+aPD-1)

14) Vector Targeting GPC3 and Inducibly Secreting IL-12 (p40-35) and Constitutively Secreting aPD-1:

EF1 a promoter-CD8α signal peptide-aGPC3scfv-CD8 hinge-CD8 transmembrane region-4-1BB-6XNFAT promoter-IgG kappa signal peptide-IL-12 (p40-35)-P2A-IgG kappa signal peptide-aPD-1 scfv (aGPC-3BB+iIL-12-P2A+aPD-1)

The above sequences are synthesized and cloned into the lentiviral expression plasmid pLenti, and verified by sequencing. The nucleic acid and amino acid sequences corresponding to each part are shown in the table below.

| | Nucleic acid | Aminoa cid |
|---|---|---|
| | sequence | sequence |
| | SEQ ID NO: | SEQ ID NO: |
| EF1apromoter | 1 | / |
| PGK promoter | 2 | / |
| 6XNFAT promoter | 3 | / |
| IL-2 promoter | 4 | / |
| IgG kappasignal peptide | 5 | 6 |
| SP-Kappa 128477 | 7 | 8 |
| CD8 hinge region and transmembrane region | 9 | 10 |
| CD3Z signal domain | 11 | 12 |
| 4-1BB signal domain | 13 | 14 |
| IL-2 | 15 | 16 |
| IL-12(P40-35) | 17 | 18 |
| IL-15 | 19 | 20 |
| IL-18 | 21 | 22 |
| aGPC3scfv | 23 | 24 |
| aPD-1 scfv | 25 | 26 |
| aPD-L1 scfv | 27 | 28 |
| P2A | 29 | 30 |
| aPSMA scfv | 31 | / |
| aPSMA BB | 32 | / |
| aPSMA BBZ | 33 | / |
| aPSMA BB + ilL-12 | 34 | / |
| aPSMABBZ + ilL-12 | 35 | / |
| aPSMA BB + ilL-15 | 36 | / |
| aPSMABBZ + ilL-15 | 37 | / |
| aPSMA BB + ilL-18 | 38 | / |
| aPSMA BB-ilL-2 | 39 | / |
| aPSMABB-IL-12 | 40 | / |
| aGPC-3BB | 41 | / |
| aGPC-3BBZ | 42 | / |
| aGPC-3BB + iaPD-1 | 43 | / |
| aGPC-3BBZ + iaPD-1 | 44 | / |
| aGPC-3 BB + iaPD-L1 | 45 | / |
| aGPC-3BBZ + iaPD-L1 | 46 | / |
| aGPC-3BB-PGK + aPD-1 | 47 | / |
| aGPC-3BB-P2A + aPD-1 | 48 | / |
| aGPC-3BB + ilL-2 | 49 | / |
| GPC-3BB-PGK + IL-12-P2A + aPD-1 | 50 | / |
| aGPC-3BB + ilL-12-P2A + aPD-1 | 51 | / |
| aCD19 scfv | 52 | 53 |
| aCD19BBZ | 54 | 55 |
| aMUC16 scfv | 56 | 57 |
| aFAP scfv | 58 | 59 |
| OX40 | 60 | 61 |
| aPD-1 HCDR1 | / | 62 |
| aPD-1 HCDR2 | / | 63 |
| aPD-1 HCDR3 | / | 64 |
| aPD-1 LCDR1 | / | 65 |
| aPD-1 LCDR2 | / | 66 |
| aPD-1 LCDR3 | / | 67 |
| aPD-L1 HCDR1 | / | 68 |
| aPD-L1 HCDR2 | / | 69 |
| aPD-L1 HCDR3 | / | 70 |
| aPD-L1 LCDR1 | / | 71 |
| aPD-L1 LCDR2 | / | 72 |
| aPD-L1 LCDR3 | / | 73 |
| aPSMA HCDR1 | / | 74 |
| aPSMA HCDR2 | / | 75 |
| aPSMA HCDR3 | / | 76 |
| aPSMA LCDR1 | / | 77 |
| aPSMA LCDR2 | / | 78 |
| aPSMA LCDR3 | / | 79 |
| aGPC3 HCDR1 | / | 80 |
| aGPC3 HCDR2 | / | 81 |
| aGPC3 HCDR3 | / | 82 |
| aGPC3 LCDR1 | / | 83 |
| aGPC3 LCDR2 | / | 84 |
| aGPC3 LCDR3 | / | 85 |
| aCD19 HCDR1 | / | 86 |
| aCD19HCDR2 | / | 87 |
| aCD19HCDR3 | / | 88 |
| aCD19HCDR4 | / | 89 |
| aCD19 LCDR1 | / | 90 |
| aCD19LCDR2 | / | 91 |
| aCD19LCDR3 | / | 92 |
| aMUC16HCDR1 | / | 93 |
| aMUC16HCDR2 | / | 94 |
| aMUC16HCDR3 | / | 95 |
| aMUC16LCDR1 | / | 96 |
| aMUC16LCDR2 | / | 97 |
| aMUC16LCDR3 | / | 98 |
| aFAP HCDR1 | / | 99 |
| aFAP HCDR2 | / | 100 |
| aFAP HCDR3 | / | 101 |
| aFAP LCDR1 | / | 102 |
| aFAP LCDR2 | / | 103 |
| aFAP LCDR3 | / | 104 |
| aGPC3 VH | / | 105 |
| aGPC3 VL | / | 106 |
| aPSMA VH | / | 107 |
| aPSMA VL | / | 108 |
| aPD-1 VH | / | 109 |
| aPD-1 VL | / | 110 |
| aPD-L1 VH | / | 111 |
| aPD-L1 VL | / | 112 |
| aCD19 VH | / | 113 |
| aCD19 VL | / | 114 |
| aMUC16 VH | / | 115 |
| aMUC16 VL | / | 116 |
| aFAP VH | / | 117 |
| aFAP VL | / | 118 |
| aEGFR VH | / | 119 |
| aEGFR VL | / | 120 |
| aHER2 VH | / | 121 |
| aHER2 VL | / | 122 |
| aCD3 VH | / | 123 |
| aCD3 VL | / | 124 |
| aFAP scfv-aCD3 | / | 125 |
| aHER2-aCD3 | / | 126 |
| aCD3 HCDR1 | / | 127 |
| aCD3 HCDR2 | / | 128 |
| aCD3 HCDR3 | / | 129 |
| aCD3 LCDR1 | / | 130 |
| aCD3 LCDR2 | / | 131 |
| aCD3 LCDR3 | / | 132 |
| aEGFR HCDR1 | / | 133 |
| aEGFR HCDR2 | / | 134 |
| aEGFR HCDR3 | / | 135 |
| aEGFR LCDR1 | / | 136 |
| aEGFR LCDR2 | / | 137 |
| aEGFR LCDR3 | / | 138 |
| aHER2 HCDR1 | / | 139 |
| aHER2 HCDR2 | / | 140 |
| aHER2 HCDR3 | / | 141 |
| aHER2 LCDR1 | / | 142 |
| aHER2 LCDR2 | / | 143 |
| aHER2 LCDR3 | / | 144 |
| CD28 intracellular signaltransduction | 145 | 146 |
| domain | | |
| LAG3 intracellular signaltransduction | 147 | 148 |
| domain | | |
| NF-_{K}bresponse element.1 | 149 | / |
| NF-_{K}bresponse element.2 | 150 | / |
| NF-_{K}bresponse element.3 | 151 | / |
| NF-_{K}bresponse element.4 | 152 | / |
| NF-_{K}bbasal promoter.1 | 153 | / |
| NF-_{K}bbasal promoter.2 | 154 | / |
| NF-_{K}bbasal promoter.3 | 155 | / |
| NF-_{K}b response promoter | 156 | / |

### Embodiment 2: Lentiviral Package and Preparation of CAR-T Cells

The Lentiviral packaging cell line 293T cells were seeded in a 10 cm cell culture dish pre-coated with poly-L-lysine, and transfected with the confluence rate of 70%-80% at 37° C., 5% CO₂. The lentiviral expression plasmid prepared in Embodiment 1 and helper plasmids psPax2 and pMD2.G were co-transfected into 293T cells with transfection reagent PEI in a mass ratio of 4:3:1, and further cultured at 37° C., 5% CO₂. After 8-12 hours, supernatant was removed and fresh medium added. Supernatant was collected after further 48 hours' culture and centrifuged at 4° C., 2000 rpm for 10 minutes to remove cell debris, the supernatant was further filtered with a 0.45 μm sterile filter and placed in a low-temperature refrigerator at −80° C. for storage or directly used for T cell infection.

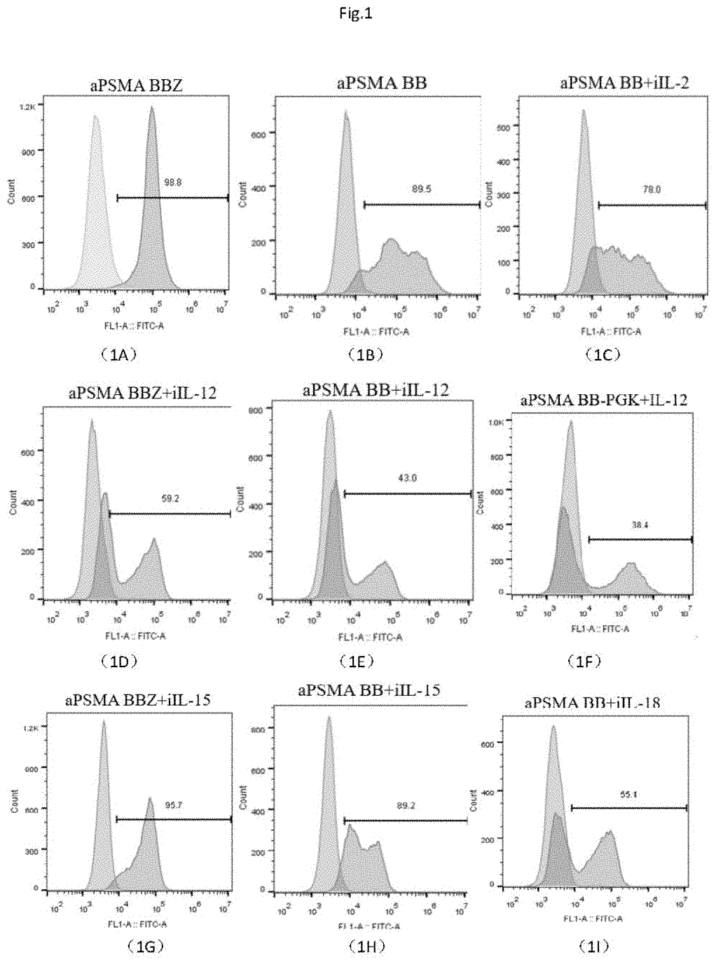

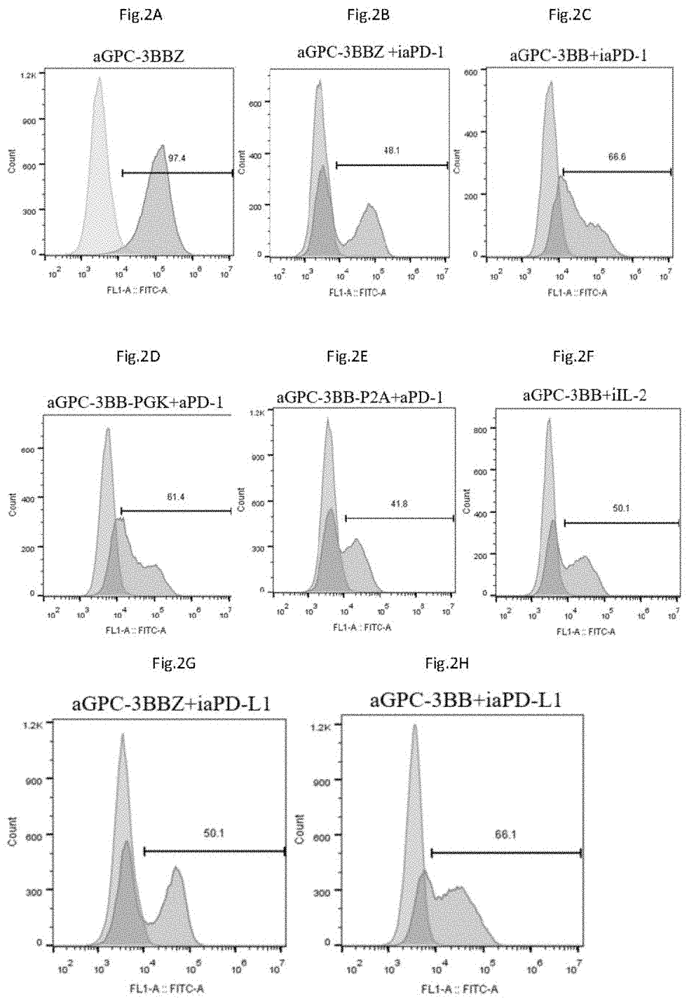

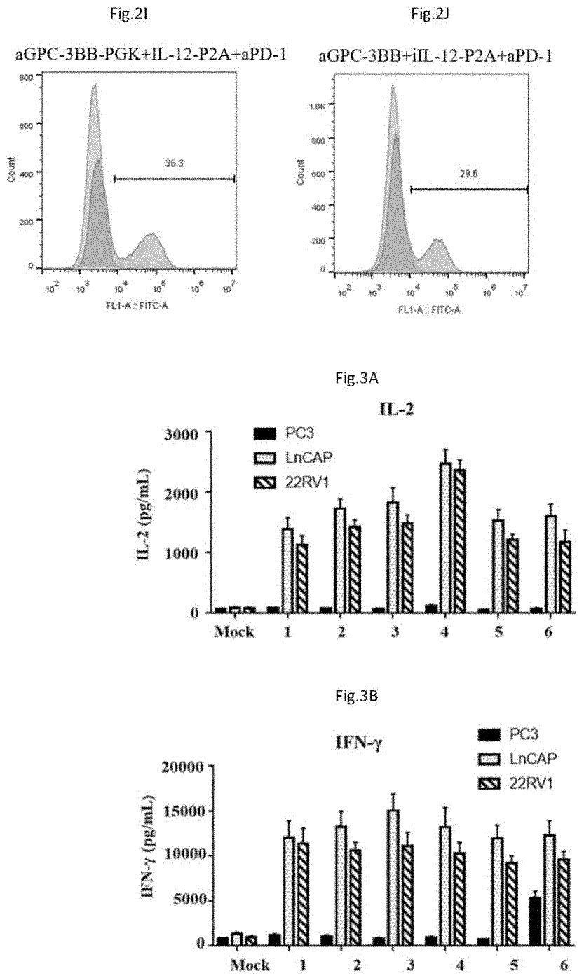

Peripheral blood mononuclear cells (PBMCs) were isolated from healthy donors with lymphocyte separation solution, transferred to a cell culture flask and cultured at 37° C., 5% CO₂ for 1 hour to remove adherent cells. Suspended PBMCs were adjusted to 2×10⁶/mL and activated for 2 days with anti-CD3 antibody OKT-3 and anti-CD28 antibody 15E8. After activation, an appropriate number of cells were mixed with fresh or thawed virus solution as mention above as well as with protamine at a final concentration of 8 μ/mL. The mixed cells were subjected to centrifuge at 32° C., 1000×G for 1 hour in a horizontal rotor centrifuge(the centrifuge was set with increase speed of 9 and a decrease speed of 0). After discarding the supernatant, cells were further cultured for 8 hours and transferred to fresh medium containing 300 IU/mL IL-2. 72 hours later, CAR expression on cells was detected by flow cytometry. Results were shown in FIG. 1 and FIG. 2A-2J.

### Embodiment 3: In Vitro Killing Activity of PSMA-Targeting CART Cells With Secretory Pro-Inflammatory Factor

### 3.1 Secretion of IL-2, IFN-γ, IL-12 and IL-18 By PSMA-Targeting CAR-T Cells

Effector cells (such as PSMA-targeted CART with secretory functions in the embodiment) and target cells (such as PC3, LnCAP or 22RV1 cells) are mixed in a 12-well plate at a ratio of 1:1 (both the cell number of the effector cells and target cells in each well was 1*10⁶. After incubating at 37° C., 5% CO₂ for 24 hours, the culture supernatant was collected, and the levels of IL-2, IL-12, IFN-γ and IL-18 in the supernatant were detected with Human IL-2 ELISA MAX™ Standard (BioLegend), Human IL-12 ELISA MAX™ Standard (BioLegend), HUMAN IFN GAMMA ELISA RSG (eBioscience) and human interleukin 18 (IL-18) ELISA kits, respectively.

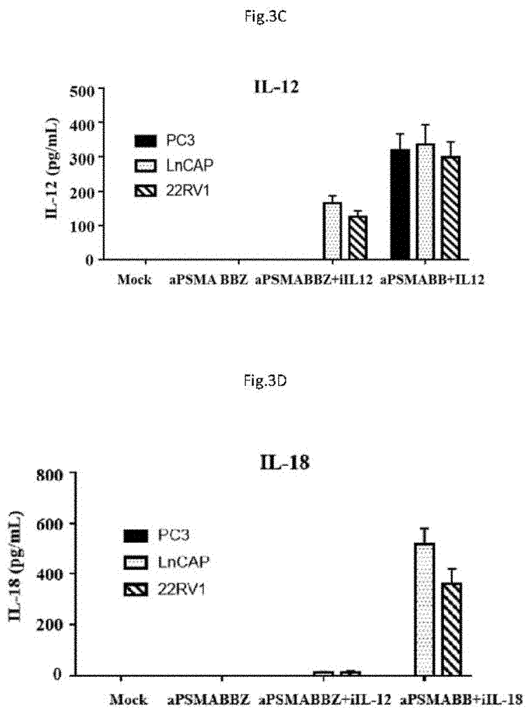

Results were shown in FIG. 3A-3D. aPSMA-expressing CART cells could effectively secret IL-2 and IFN-γ when incubated with PSMA-positive target cells LnCAP or 22RV1,while no IL-2 or IFN-γ secretion was detected when incubated with PSMA-negative target cells PC3. The cytokines secretion of the CART cells (cells expressing aPSMA BB+iIL-2, aPSMA BB+iIL-18 or aPSMA BB+IL-12) lacking an artificial first signal CD3Z were similar to that of CART cells (cells expressing aPSMA BBZ, aPSMA BBZ+iIL-12, aPSMA BBZ+iIL-15) containing the artificial first signal CD3Z (FIGS. 3A-3D).

### 3.2 In Vitro Killing Activity of PSMA-Targeting CAR-T Cells

The effector cells (such as PSMA-targeting CART cells with secretory cytokines in the embodiment) and target cells (such as PC3 or LnCAP cells) were incubated at 37° C., 5% CO₂ at different ratios (20:1, 10:1, 5:1, 2:1, 1:1, and 0.5:1). After 24 hours, cell supernatant was collected, and the levels of LDH was detected by Cyto Tox 96 Non-Radioactive Cytotoxicity Assay (Promega).

Results were shown in FIG. 5, CART cells expressing aPSMA BBZ or aPSMA BBZ+iIL-12 could effectively exert killing activity on PSMA-positive LnCAP cells while not on PMSA-negative PC3 cells.

### Embodiment 4: In Vitro Killing Activity of GPC3-Targeted CART Cells With Secretory Immune-Checkpoint Inhibitors

### 4.1 Detection of IL-2 and aPD-1 scfv Secretory By CAR-T Cells

GPC3-targeting effector cells (such as CART cells) and target cells (such as A431 or HepG2 cells) were incubated at a ratio of 1:1 at 37° C., 5% CO₂ After 24 hours, cell supernatant was collected, and the level of IL-2 in the supernatant was detected with Human IL-2 ELISA MAX™ Standard (BioLegend).

The aPD-1 scfv expression was detected by sandwich ELISA: hPD-1 was coated on a 96-well plate, and incubated at 4° C. overnight. After blocking with PBST (0.5% Tween-20 in phosphate buffered saline (PBS)) containing 2% fetal bovine serum (FBS) at room temperature for 1 hour, the cell supernatant above was added, and then incubated at room temperature for 2 h. After being washed for 4-5 times with PBST containing 2% FBS, goat anti-Human IgG(Fab′)2(HRP) secondary antibody was added and incubated at room temperature for 1 h, followed by washing with PBST containing 2% nonfat dry milk for 4-5 times. Reading at OD450 nm were recorded after TMB reagent (BioLegend) was added for color development.

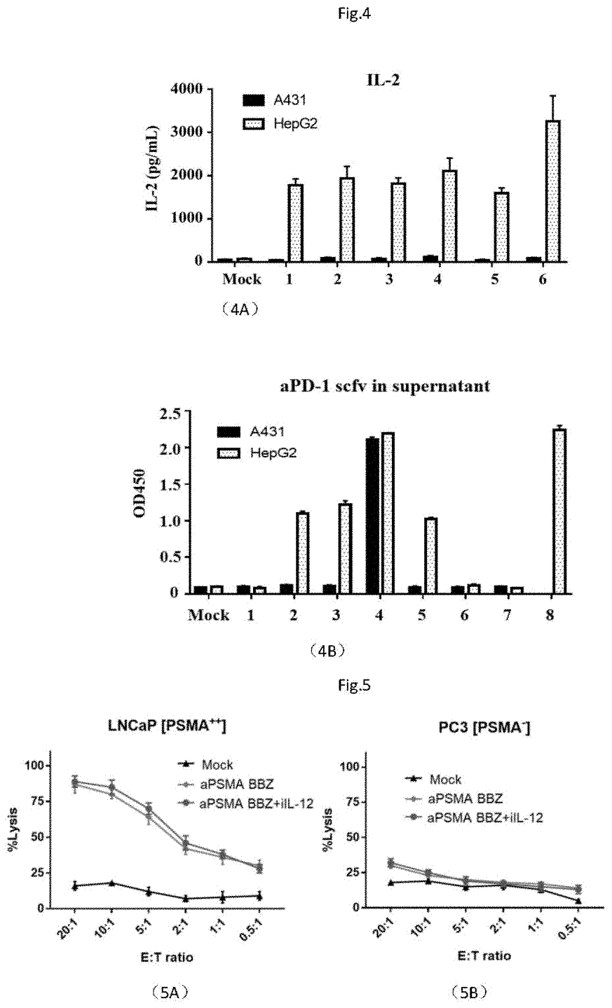

Results were shown in FIG. 4. The level of IL-2 secretion by CART cells lacking an artificial first signal CD3Z (aGPC-3BB+iaPD1, aGPC-3BB+iaPD-L1, aGPC-3BB-PGK+aPD-1, aGPC-3BB-P2A+iaPD-1, and aGPC-3BB+iIL-2) was similar to that of IL-2 secretory by CART cells containing the artificial first signal CD3Z when incubating with GPC-3 positive target cells HepG2. CART cells lacking the artificial first signal CD3Z while constitutively expressing aPD-1 scfv showed significantly higher level of aPD-1 scfv expression (aGPC-3BB-PGK+aPD-1), compared to CART constructs with inducible a PD-1 scfv secretion (aGPC-3BB+iaPD1, aGPC-3BBZ+iaPD-1, and aGPC-3Z+iaPD-1).

### 4.2 In Vitro Killing Activity of GPC-3-Targeting CAR-T Cells

Effector cells (such as GPC-3-targeting CART cells in the embodiment) and target cells (such as HepG2 or A431 cells) were co-incubated at 37° C., 5% CO₂at different ratios (5:1, 3:1, 1:1, 1:3, and 1:5). After 24 hours, cell supernatant was collected, and the LDH level was detected with CytoTox 96 Non-Radioactive Cytotoxicity Assay (Promega).

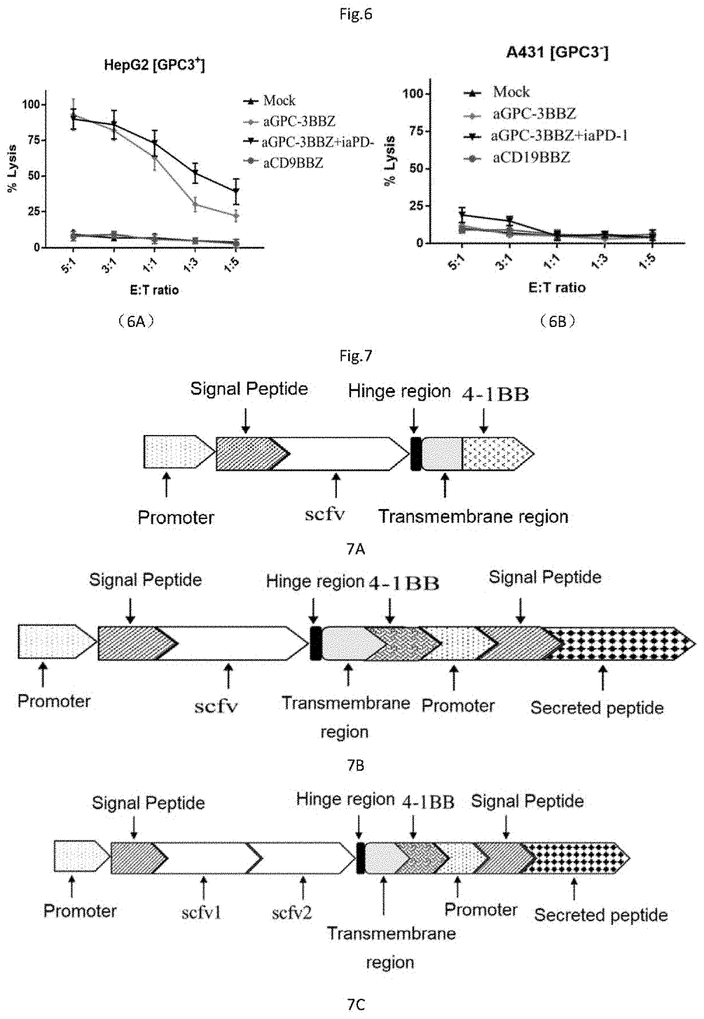

Results were shown in FIG. 6, CART cells expressing aGPC-3 BBZ or aGPC-3 BBZ+iaPD-1 could effectively exert killing activity on GPC-3 positive HepG2 cells while not on GPC-3 negative A431 cells.

### Embodiment 5: In Vitro Killing Activity of PSMA-Targeting CART Cells

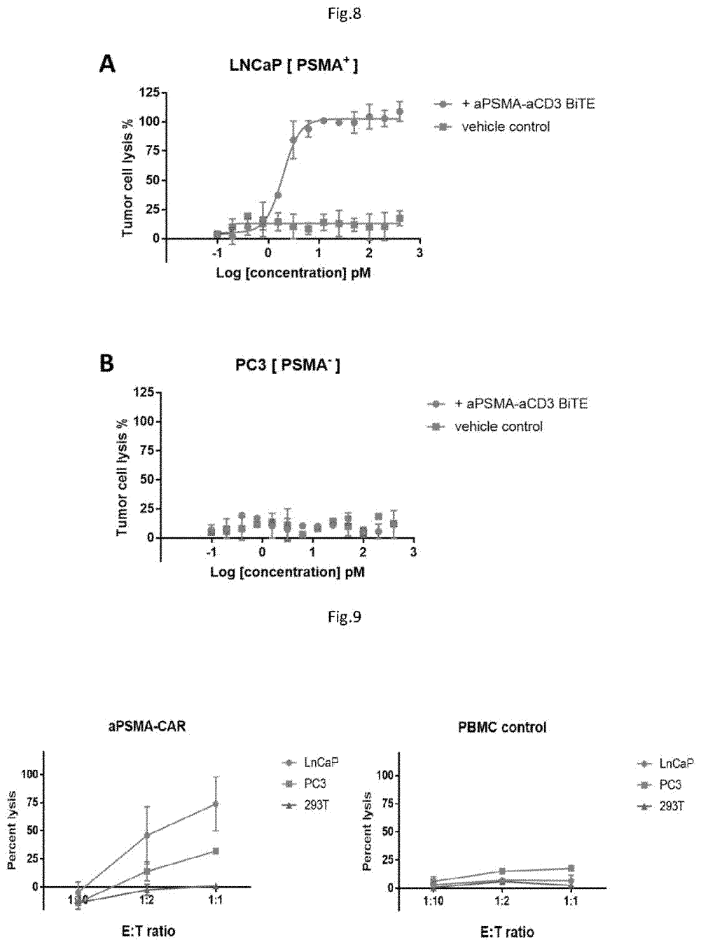

aPSMA BBZ CAR-T cells were incubated with two target cells (LNCaP and PC3) at a ratio of 1:1 (10⁴ cells each per well) in a 96-well plate(flat bottom), respectively. Bispecific antibody aPSMA-aCD3 BiTE pre-diluted in a two-fold gradient and PBS (as solvent control)were added, and incubated in a cell incubator (37° C., 5% CO₂) for 16 hours. CytoTox 96° Non-Radioactive Cytotoxicity Assay (Promega™) kit was used to detect the killing activity of CAR-T cells on target cells. As shown in FIG. 8, PSMA-specific CAR-T cells containing only costimulatory signal transduction domain showed specific killing activity on PSMA-positive tumor target cells LnCaP while not on PSMA-negative tumor target cells PC3 in the presence of first activation signal mediated by aPSMA-aCD3 BiTE molecules.

CAR-T effector cells derived from patient's PBMCs and target cells (such as tumor cells, PC3 cells or LnCAP cells) were mixed at different ratios (1:10, 1:2, 1:1), herein the number of the target cells was 10⁴ cells/well. After incubation at 37° C., 5% CO₂ for 24 hours, cell culture supernatant was collected, and LDH level was detected by CytoTox 96 Non-Radioactive Cytotoxicity Assay (Promega) kit. Results were shown in FIG. 9, patient-derived CART cells expressing aPSMA BBZ could specifically and effectively exert killing activity on PSMA-positive tumor cells in vitro. In contrast, activated T cells without CAR transfection exhibited no killing activity.

### Embodiment 6: NF-κb Responsive Promoter Induced By Costimulatory Signal Activation

Firefly luciferase reporter gene elements driven by NF-κb responsive promoter (from N-terminal to C-terminal: NF-κb responsive promoter-firefly luciferase reporter gene-PGK promoter-green fluorescent protein) were introduced into PBMC cells by lentiviral transduction. The prepared PBMC cells were plated in a 96-well plate at a density of 4×10⁴ cells/well, and stimulants (TNFα, Phorbol myristate acetate, 4-1bbL) pre-diluted in a two-fold gradient and PBS (as solvent control) were further added. After incubation (37° C., 5% CO₂) for 6 hours, fluorescence in well was detected by Firefly Luc One-Step Glow Assay Kit (Pierce™), and the induction of luciferase expression were calculated with the solvent control as the base line.

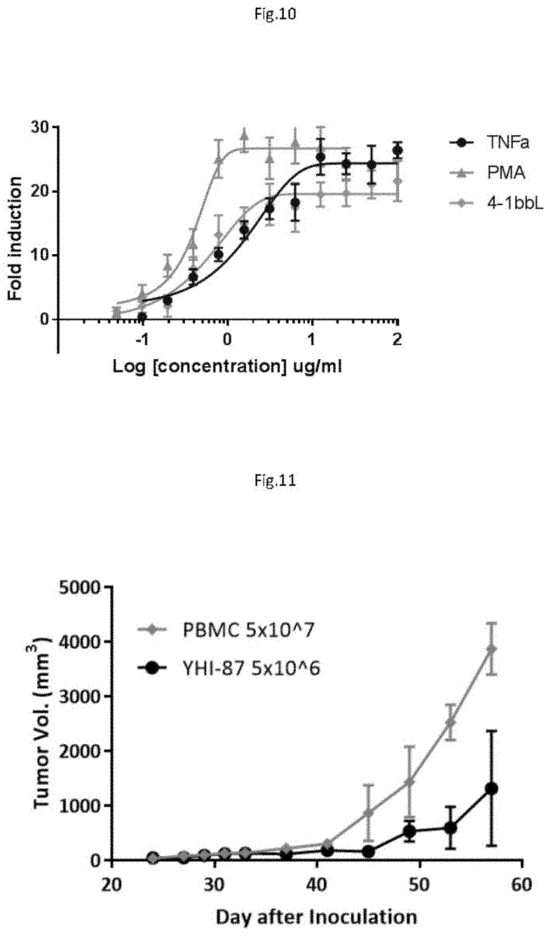

As shown in FIG. 10, TNFα, Phorbol myristate acetate (PMA) and 4-1bbL could all activate the NF-κb responsive promoter in PBMC cells and drive the expression of downstream genes.

### Embodiment 7: In Vivo Study With Mouse Model

Efficacy studies of PSMA-targeted CAR were performed in 6-week-old male NCG mice. 2×10⁶LnCap cells were subcutaneously(SC) injected into the right flank of mice, and the size of tumor was measured with calipers three times a week. When the volume of the tumor reached 100-200 mm³, CART cells were intravenously injected into the tail (Day 0) (The experiments were divided into two groups: activated T cell injection group, and 5×10⁶ CART cell injection group). Tumor size and mouse body weight were measured weekly. The tumor volume was calculated by the following formula: tumor volume=width length*height/2. Five weeks after CART injection, the mice are sacrificed, serum or plasma was collected for cytokine or immune factor analysis and mouse tissues were collected. CART cell population in TILs and PBMCs were analyzed by flow cytometry and immunohistochemistry. For CARTs with secretory function, the levels of the secretory proteins in tumor and blood were further analyzed.

As were shown in FIG. 11, CART cells expressing aPSMA BBZ exhibited efficient killing activity to PSMA-positive solid tumors in mice, and the activated T cells without CAR transfection at a dose up to 5×10⁷showed no killing activity.

1. Chimeric antigen receptor (CAR)-modified T (CART) cells for use in cancer treatment, wherein the CART cells comprise a first CAR, wherein the first CAR comprises an antigen binding domain, a transmembrane domain and an intracellular domain, wherein the intracellular domain contains artificially introduced costimulatory signal transduction domains, wherein the CART cells does not contain an artificially introduced first signal transduction domain.

2. The chimeric antigen receptor (CAR)-modified T (CART) cells for use in cancer treatment according to claim 1, wherein the artificially introduced costimulatory signal transduction domain is selected from one or more of the following intracellular signal transduction domains of costimulatory proteins: 4-1BB (CD137), CD28 , CD27, CD30, OX40, GITR, CD40, BAFFR, ICAM-1, LCK, CD278(ICOS), CD150(SLAMF1), CD270(HVEM), LAT, NKD2CSLP76, TRIM, ZAP70, DAP-10, DAP-12, LFA-1, CD2, CDS, CD7, CD287, LIGHT, NKG2C, NKG2D, SLAMF7, NKp80, NKp30, NKp44, NKp46, CD160, B7-H3 or a ligand that specifically binds to CD83 and the like.

3. The chimeric antigen receptor (CAR)-modified T (CART) cells for use in cancer treatment according to claim 2, wherein the artificially introduced costimulatory signal transduction domain is an intracellular signal transduction domain of 4-1BB or OX40.

4. The chimeric antigen receptor (CAR)-modified T (CART) cells for use in cancer treatment according to claim 1, wherein the antigen binding domain binds to disease-associated cell surface antigens.

5. The chimeric antigen receptor (CAR)-modified T (CART) cells for use in cancer treatment according to claim 4, wherein the disease-associated cell surface antigen is selected from immune checkpoint proteins or tumor antigens.

6. The chimeric antigen receptor (CAR)-modified T (CART) cells for use in cancer treatment according to claim 1, wherein the CART cell further expresses a second CAR and/or secretory polypeptides, wherein the second CAR comprises an antigen binding domain and a transmembrane domain, wherein the secretory polypeptides are constitutively or inducibly expressed.

7. The chimeric antigen receptor (CAR)-modified T (CART) cells for use in cancer treatment according to claim 6, wherein the antigen binding domain of the second CAR binds to disease-associated cell surface antigens, and preferably, the disease-associated cell surface antigen is a tumor antigen.

8. The chimeric antigen receptor (CAR)-modified T (CART) cells for use in cancer treatment according to claim 7, wherein the secretory polypeptides are selected from but not limited to: one or two of the followings, immune function regulatory factors, antibodies specifically targeting the tumor antigen, or antibodies specifically targeting the immune checkpoint.

9. The chimeric antigen receptor (CAR)-modified T (CART) cells for use in cancer treatment according to according to claim 5, wherein the tumor antigen is independently selected from: epidermal growth factor receptor family (EGFR, HER2, HER3, HER4), PD-1, PD-L1 , CTLA-4, 4-1BB(CD137), OX40, CD28, CD40, CD47, CD70, CD80, CD122, GTIR, A2AR, B7-H3(CD276), B7-H4, IDO, KIR, Tim-3, NY-ESO-1, GPC3, CLL-1, BCMA, mucin family (MUC1, MUC2, MUC3A, MUC3B, MUC4, MUC5AC, MUCSB, MUC6, MUC7, MUC8, MUC12, MUC13, MUC15, MUC16, MUC17, MUC19, MUC20), CD19, CD20, CD22, CD30, CD33, CD52, chemokine receptor family (CCR1, CCR2, CCR3, CCR4, CCR5, CCR6, CCR7, CCR8, CCR9, CCR10, CCL27, CCL28, CX3CR1, CXCR1, CXCR2, CXCR3, CXCR4, CXCR5, CXCR6), PSMA, CEA, HDAC6, EpCAM, Mesothelin, TERT, TLR, TLR9, TLR4, CD33, GITR, Survivin, CD123, TIGIT, TIM-3, CD73, fibroblast growth factor body (FGFR), vascular endothelial growth factor receptor (FLT1, KDR/Flk-1, VEGFR-3), hepatocyte growth factor receptor (HGFR), nerve growth factor receptor (NGFR), insulin-like growth factor receptor (IGFR), platelet-derived growth factor receptor (PDGFR) or hormone receptor (melanocortin 1 receptor (MC1R, MSHR)) and the like.

10. The chimeric antigen receptor (CAR)-modified T (CART) cells for use in cancer treatment according to claim 9, wherein the tumor antigen is PSMA, GPC3, CD19, MUC16, EGFR, HER2, CD3 or FAP.

11. The chimeric antigen receptor (CAR)-modified T (CART) cells for use in cancer treatment according to claim 5, wherein the immune checkpoint protein is selected from: PD-1, PD-L1, CTLA-4, LAG-3, OX40, CD28, CD40, CD47, CD70, CD80, CD122, GTIR, A2AR, B7-H3(CD276), B7-H4, IDO, KIR, Tim-3 or 4-1BB (CD137).

12. The chimeric antigen receptor (CAR)-modified T (CART) cells for use in cancer treatment according to claim 11, wherein the immune checkpoint protein is PD-1 or PD-L1.

13. The chimeric antigen receptor (CAR)-modified T (CART) cells for use in cancer treatment according to claim 8, wherein the immune function regulatory factor is selected from: IL-2, IL-12, IL-7, IL-15, IL-18, IL-21, IL-24, 4-1BBL, PD-1 extracellular region, PD-L1 extracellular region, PD-1 mutant, CCL19, MIP-1α, GM-CSF, IFN-α, IFN-β, IFN-γ, TNF-α, M-CSF, TGF-β or TRAIL and the like.

14. The chimeric antigen receptor (CAR)-modified T (CART) cells for use in cancer treatment according to claim 13, wherein the immune function regulatory factor is IL-2, IL-12, IL-15, IL-18, or PD-1 mutant.

15. The chimeric antigen receptor (CAR)-modified T (CART) cells for use in cancer treatment according to claim 6, wherein the secretory polypeptides are on the same peptide chain as the first or second CAR, and the secretory polypeptides are linked to the CAR by self-cleaving peptides, or the coding sequence of the secretory polypeptides are linked to the coding sequence of the CAR by promoter and signal peptide coding sequences.

16. The chimeric antigen receptor (CAR)-modified T (CART) cells for use in cancer treatment according to claim 6, wherein the secretory polypeptides are on different peptide chains from the first or second CAR.

17. The chimeric antigen receptor (CAR)-modified T (CART) cells for use in cancer treatment according to claim 5, wherein the antigen binding domain is an antibody or an antibody fragment.

18. The chimeric antigen receptor (CAR)-modified T (CART) cells for use in cancer treatment according to claim 6, wherein the secretory polypeptides are proteins, antibodies, or antibody fragments.

19. The chimeric antigen receptor (CAR)-modified T (CART) cells for use in cancer treatment according to claim 1, wherein the T cells is selected from one or more subsets of specific T cells, such as a tumor-infiltrating lymphocyte (TIL), a cytotoxic T lymphocyte (CTL), a natural killer T (NKT) cell, a δ T cell or a regulatory T cell.
